(19) United States
(12) Patent Application Publication
(10) Pub. No.: US 2005/0169928 A1
(43) Pub. Date: Aug. 4, 2005

(54) Antibodies which bind to proteins involved in cytoadhesion of plasmodlum falciparum ring-stage-infected erythrocytes

(75) Inventors: Juerg Gysin, Saint Zacharie (FR); Arthur Scherf, Paris (FR); Catherine Lepolard, Aubagne (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: INSTITUT PASTEUR, Paris (FR); Centre National De La Recherche Scient., Paris Cedex (FR)

(21) Appl. No.: 11/095,534

(22) Filed: Apr. 1, 2005

Related U.S. Application Data
(63) Continuation of application No. 10/358,334, filed on Feb. 5, 2003, now abandoned.
Application No. 10/358,334 is a continuation-in-part of application No. 09/867,536, filed on May 31, 2001, now abandoned.
(60) Provisional application No. 60/207,952, filed on May 31, 2000.

(51) Int. Cl. A61K 39/395; C07K 16/20

(52) U.S. Cl. 424/151.1; 530/388.6

(57) ABSTRACT

The present invention provides antibodies that specifically bind RSP-2 proteins which are involved in the cytoadhesion of P. falciparum during ring-stage infection of erythrocytes as well as methods of using these antibodies.

### CROSS-REFERENCE TO RELATED APPLICATIONS

This application claims priority to U.S. Provisional Application Ser. No. 60/207,952 filed May 31, 2000, which is incorporated herein by reference and is a continuation-in-part application of U.S. Ser. No. 09/867,936 filed May 31, 2001, which is pending.

### BACKGROUND OF THE INVENTION

1. Field of the Invention

The present invention provides antibodies that specifically bind RSP-2 proteins which are involved in the cytoadhesion of P. falciparum during ring-stage infection of erythrocytes as well as methods of using these antibodies.

2. Description of the Background

A common pathological characteristic in P. falciparum infection is the cytoadhesion of mature-stage infected erythrocytes (MIE) to host endothelium and syncytiotrophoblasts. Massive accumulation of IE in the brain microvascular or placenta is strongly correlated with severe form of malaria (Miller, L. H., Good, M. F. & Milton, G. Malaria pathogenesis. Science 264, 1878-1883 (1994)). Extensive binding of IE to placental CSA is associated with physiopathology during pregnancy (Fried, M. & Duffy, P. E. Adherence of Plasmodium falciparum to chondroitin sulfate A in the human placenta. Science 272, 1502-1504 (1996); Gysin, J., Pouvelle, B. Fievet, N., Schert, A. & Lepolard, C. Ex vivo desequestration of Plasmodium falciparum-infected erythrocytes from human placenta by chondroitin sulfate A. Infect. Immun. 67, 6596-6602 (1999)). The adhesive phenotype of IE correlates with the appearance of PfEMP1 at the erythrocyte surface (approx. 16 hours after merozoite invasion) and therefore only early blood-stage (ring-stage) IE are seen in the peripheral blood. Here we describe results that challenge the existing view of blood-stage IE biology. We demonstrate the specific adhesion of IE, during the early ring-stage, to endothelial cell lines from brain and lung and to placental syncytiotrophoblasts. Later in the blood-stage development of these IE, trophozoites switch to an exclusively chondroitin-sulphate A (CSA) cytoadhesion phenotype. Therefore, adhesion to an individual endothelial cell or syncytiotrophoblast may occur throughout the blood stage cycle, suggesting that there are non-circulating (cryptic) parasite subpopulations in malaria patients. We detected two novel parasite proteins on the surface of ring-stage IE. These proteins disappear shortly after the start of PfEMP1-mediated adhesion. These data have important implications for epidemiological studies, parasite tissue tropism and malarial disease outcome.

### SUMMARY OF THE INVENTION

One object of the present invention is to provide an antibody which binds to the RSP-2 protein and in one embodiment of this object, the antibody would also have the ability to inhibit cytoadhesion of erythrocytes, which express the RSP-2 protein on the cell surface (i.e., RSP-2 positive erythrocytes), to desequester RSP-2 positive erythrocytes from endothelial cells and/or to facilitate clearance of RSP-2 positive erythrocytes from the blood of an individual infected with Plasmodium sp.

Thus, another object of the invention is to provide a method of treating a Plasmodium sp. infection in a patient by administering at least one of these antibodies to the individual so that the antibody desequesters at least a part of the RSP-2 erythrocytes from endothelial cells, and/or inhibit merozoite invasion, and/or facilitate clearance of RSP-2 positive erythrocytes by the spleen.

Another object of the present invention is to provide methods of identifying a substance by contacting the RSP-2 with the substance and determining whether the substance binds to the same epitope as those antibodies and in one aspect of this object the substance would also have the ability to the ability to inhibit cytoadhesion of erythrocytes, which express the RSP-2 protein on the cell surface (i.e., RSP-2 positive erythrocytes), to desequester RSP-2 positive erythrocytes from endothelial cells and/or to facilitate clearance of RSP-2 positive erythrocytes from the blood of an individual infected with Plasmodium sp. The substance could be, for example, another antibody or antibody fragment.

These substances could then be formulated into an appropriate composition and used for treating Plasmodium sp. infection as described herein.

### BRIEF DESCRIPTION OF THE DRAWINGS

FIG. 1: Ring-stage IE^{CSA} cytoadhesion to various cells and tissues. IE^{CSA} from highly synchronized cultures cytoadhered to (A) cryosections of liquid nitrogen snap-frozen uninfected human placenta biopsy samples (R, ring- and T, trophozoite stage) or (B) SBEC ID monolayer (Nikon E800, ×1000). (C) Cytoadhesion of synchronized PA^{CSA} IE, at the 8th hour after invasion, to monolayers of SBEC 1D, HUVEC, HLEC primo explants, HMEC, C32 and CHO cells and to cyosections of liquid nitrogen snap-frozen uninfected human placenta biopsy samples. After extensive washing, the number of cytoadherent IE per high-power fields of placental cryoscections or per mm² of cell monolayer counted on 4 random fields (0.25 mm² area at ×300 magnification, Olympus CK2) over the entire surface of each sample was determined. The values for each experiment were standarized to 5% parasitaemia, and the results expressed as a mean cytoadhesion value±SD. (D) Highly synchronized populations of PA^{CSA} and PA^{CD36} IE were mixed at the 8th hours of the cycle to give an initial population, the phenotype distribution of which was determined 24 hours later by cytoadhesion inhibition microassays. The rest of the mixture was immediately subjected to selection by cytoadhesion on SBEC 1D. The cytoadherent IE were cultured and the phenotype distribution of the result population determined. The percentage cytoadhesion obtained for each inhibitor. CSA (▪) and anti-CD36 FA6-152 monoclonal antibodies (□) gives the inverse proportion of each phenotype in the IE populations.

FIG. 2: Cytoadhesion throughout the blood-stage cycle is mediated by a switch in adhesion phentype. (A) Determination of the inhibitory activity of CSA (▪) and chondroitinase ABC (□) on the cytoadhesion of SBEC 1D of highly synchronized PA^{CSA} IE, every 4 hours throughout the cycle. (B) Cytoadhesion of ring-stage PA^{CSA} IE to SBEC ID 8 hours post-invasion in the presence of dermatan-sulphate (CSB), chondroitin-6-sulphate (CSC), keratan sulphate (Ker), hyaluronic acid (HA), heparin (Hep), or after treatment of the target cells with chondroitinase ABC (Case ABC) and B (Case B), hyaluronate lyase (H Lyase) and heparinase II (Hepase).

FIG. 3: Adhesive ring-stage IE express novel ring stage-specific surface molecules that are targets of the immune response. (A) Identification of a high-molecular weight ¹²⁵I-labeled surface antigen on a ring-stage IE. The separation of SDS extracts of the iodinated surface FCR3^{CSA} at various time points after merozoite re-invasion (7, 14, 21 and 32 hours) is shown. A single labeled band of approximately 200 kDa (RSP-1) is detected from the early ring (7 hour) to early trophozoite stages (21 hour). A second labeled band of approximately 400 kDa appears at 14 hours and is detected until the end of the cycle. ³⁵S-labeled ring-stage IE extracts show a 200 kDa band that co-migrates with RSP-1. The 200 and 400 kDa bands are not seen in control erythrocytes (data not shown). (B) A pool of immune sera from pregnant women (Cameroon) immunoprecipitated two major protease-sensitive proteins of approximately 200 and 40 kDa. Later in the life cycle, the var^{CSA} molecule (400 kDa) is immunoprecipitated. Lane 1: trophozoite stage 32 hours post-infection) followed by ¹²⁵I-labeled young ring-stage (lane 2) and trypsin (tryp) treatment before immunoprecipitation: 10 μg/ml trypsin (lane 3), 100 μg/ml trypsin (lane 4) and 100 μg/ml α-chymotrypsin (chymo; lane 5). ³⁵S-methionine labeled ring-stage IE SDS extract immunoprecipated with the serum pool (lane 6). (C) Sensitivity of cytoadshesion to the treatment ring-stage IE with different concentrations of trypsin or α-chymotrypsin. (D) Immunolabeling of ring-stage (R) and trophozoite-stage (T) PA^{CSA} IE with a pool of 5 sera from Senegalese and Cameroonian patients living in areas of endemic malaria. Antibodies at the surface of the IE were detected with an FITC-conjugated anti-human IgG and were observed by EPR microscopy (CELLscan). (E) Cytoadhesion inhibition by sera obtained from a primigravida (1), and multigravida (25 and 46) women, a child (61) and a male adult (1613). The percentage cytoadhesion was obtained by comparing the binding obtained in the presence of each serum with a control carried out with a pool of sera from volunteers who had never contracted malaria.

FIG. 4: A rhoptry-derived P. falciparum parasite molecule binds to the surface of normal and infected erythrocytes. Staining of nE and IE from FCR3^{CSA} parasites using anti-RSP-2 mAb B4 in IFA. Parasite DNA is stained with DAPI. A-D: L-IFA analysis. E-H: AD-IFA analysis. A. merozoite binding to the membrane of a nE stained with anti-RSP-2. B. surface staining of a nE (left) and rIE (right). C.—transfer of RSP-2 from merozoite to the entire erythrocyte surface; D. A mature trophozoite stage parasite is not stained by mAb B4. AD-IFA using B4 in young rIE (E.) in 26±2 h old parasites (F). in 34±2 h old schizont stage (G) and 44±2 h showing free merozoites (H).

FIG. 5: Anti-RSP-2 mAbs recognize a 42 kDa molecule. A. Western blot analysis using mAbs B4, C10 and D10. Triton X100 soluble protein extract of rIE^{CSA} were analysed. Lane 1, negative control IgG2a; lane 2, anti-RSP-2 C10; lane 3; anti-RSP-2 B4 and lane 4, anti-RSP-2 D10. B. Western blot with anti-RSP-2 mAb B4 and Triton X100 protein extracts. lane 1, nE; lane 2, rIE^{CSA}; lane 3, rIE^{CD36} and lane 4, rIE^{ICAM-1}.

FIG. 6: MAbs B4, C10 and D10 detect a 42 kDa protein that forms a low-molecular weight complex in rhoptry and on the surface of rIE. A. Immunoprecipitation of metabolic labelled S³⁵methionin Triton X100 protein extracts of rIE^{CSA} with mAbs. C10 (lane 1), B4 (lane 2), D10 (lane 3), negative control IgG2a, (lane 4) and protein G alone (lane 5). B. Immunoprecipitation of Triton X-100 extracts of surface iodinated FCR3^{CSA} and FCR3^{CD36} rIE using mAb B4. Lane 1: surface iodinated FCR3^{CSA} rIE immunoprecipitated by B4, Lane 2: FCR3^{CD36} immunoprecipitated by B4, Lane 3: FCR3^{CSA} immunoprecipitated by a pool of immune sera from pregnant women (Senegal), Lane 4: FCR3^{CSA}, trypsin treatment (100 μg/ml) before immunoprecipitation by B4, Lane 5: FCR3^{CD36}, trypsin treatment before immunoprecipitation by B4, Lane 6: FCR3^{CSA}, trypsin treatment before immunoprecipitation by a pool of immune sera from pregnant women (Senegal), Controls: FCR3^{CSA} immunoprecipitation with no mAbs (Lane 7) and by anti-PfEMP-1 (Lane 8).

FIG. 7: Inhibition of cytoadhesion of FCR3 rIE^{CSA} to endothelial cells using mAbs anti-RSP-2. A. Inhibition of rIE^{CSA} cytoadhesion in presence of different concentrations of mAbs B4, C10, D10. B. Inhibition of rIE^{CSA} cytoadhesion of laboratory strains from different geographical areas, FCBR, BXII, SUK, HB, IBR to SBEC 1D and (C.) placental isolates from Camerounian women, 24, 42, 42DJ, 193, 939 by anti-RSP-2 mAb B4. D. Inhibition of rIE^{CSA} cytoadhesion to Sc1D by different concentrations of mAb B4 mixed together with 2.10⁷ rIE^{CSA} under flow conditions at 0.05 Pa. Results are expressed as mean±SD % inhibition of control.

FIG. 8: Desequestration of cytoadherent rIE^{CSA} to SBEC 1D under flow conditions. Cytoadhesion was performed by flowing 2×10⁷ rIE^{CSA} over a confluent monolayer of SBEC ID grown in microslides at 0.05 Pa before rinsing at various shear stresses with culture medium. Results of residual cytoadherent rIE^{CSA} are expressed as mean±SD % inhibition of control for each shear stress increment starting at 0.05 Pa.

FIG. 9: Expression profile of RSP-2 and PfEMP1 at the erythrocyte surface during the blood stage cycle. RSP-2^{CSA} was stained with mAb B4 (green) and PfEMP1^{CSA} with mAb 1B4/D4 (red) by L-IFA on IE. Parasite nuclei are stained by DAPI (blue). A. Positive anti-RSP-2 staining on synchronised rIE (<16 hours post-invasion) and early trophozoites (16 to 20 hours after post-invasion). No surface staining was detectable on mature forms (>20 hours). B. Absence of PfEMP1 staining on rings (<16 hours) but strong IFA signal with early trophozoite and mature stages. C. Superposition of anti RSP-2 and anti-PfEMP1 staining shows co-localisation in early trophozoite stages.

FIG. 10: Blocking of rIE^{CSA} and mature IE^{CSA} adhesion to Sc1D in the presence of specific inhibitors. A culture of rIE^{CSA} and mature IE^{CSA} parasites (equal parasitemia) was passed over Sc1D in microslides at 0.05 Pa. A. A confluent endothelial cell monolayer shows adherent rIE^{CSA} and mature IE^{CSA}. B. Flushing with 2 ml of 100 μg/ml anti-RSP-2 mAb B4 in culture medium. C. Flushing with 2 ml of 100 μg/ml CSA dissolved in culture medium. D. nE, rIE^{CSA} and mature IE^{CSA} were desequestrated using both inhibitors.

FIG. 11: Inhibition of merozoite invasion using anti-RSP-2 mAbs A suspension of synchronised mature IE of the adhesion phenotypes CSA, CD36 and ICAM-1 in 200 μl/well at a final hematocrit of 2% in culture medium were incubated in 96 well flat bottom plates together with 10 μl of mAbs B4 (A.) and D10 (B.) at different concentrations. 4 h after invasion 100 μCi of ³H-hypoxanthine was added and the incorporated radioactivity counted 24 h later.

FIG. 12: Schematic model of the different types of RSP-2 tagged nE and rIE and their adhesive tropism during the blood stage cycle. A. In parasites of the CSA-binding phenotype nE that carry RSP-2 on their surface cytoadhere to endothelial cells and placental syncytiotrophoblasts via an unknown receptor. On the other hand RSP-2 rIE and nE of the CD36 and ICAM-1 phenotypes in the circulation are removed by the spleen. RSP-2 tagged nE are potential targets marked for destruction by the host immune response in the presence of anti-RSP-2 antibodies. B. rIE of parasites selected for CSA-binding will cytoadhere and during a short period of the blood stage cycle (early trophozoites), both RSP-2 and PfEMP1 are present on the surface of IE and can bind to two distinct host receptors at the same time. These parasites are probably not at all present in the peripheral blood circulation.

### DETAILED DESCRIPTION OF THE INVENTION

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

The RSP-1 and RSP-2 proteins of this invention may be purified to substantial purity by standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982).

In addition, provided with the purified proteins of the present invention one of skill in the art will be able to obtain a amino acid sequence from which the polynucleotide sequence which encodes the RSP-1 and RSP-2 proteins can be obtained. Methods for protein sequencing and isolation of a polynucleotide sequence are known in the art and include polynucleotide amplification using primers derived from the amino acid sequence of the purified proteins. These and other methods are disclosed in Current Protocols in Molecular Biology, F. M. Ausebel et al., eds., Current Protocols, a joint venture between Greene Publishing Associates, Inc. and John Wiley & Sons, Inc. (2000) and Maniatis et al. Molecular Cloning: A Laboratory Manual, Cold Spring Harbor Press, 1988.

Having obtained the polynucleotide sequences encoding the RSP-1 and RSP-2 proteins, the polynucleotide sequences can be constructed in recombinant expression vectors for expression of the genes in transfected cells. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (2000).

Cell cultures that may be used in the present invention, include cell lines and cultured cells from tissue or blood samples is well known in the art. Freshney (Culture of Animal Cells, a Manual of Basic Technique, third edition Wiley-Liss, New York (1994)) and the references cited therein provides a general guide to the culture of cells.

Proteins produced by recombinant DNA technology may be purified by standard techniques well known to those of skill in the art. These proteins can be directly expressed or expressed as a fusion protein. The protein can then be purified by a combination of cell lysis (e.g., sonication) and affinity chromatography. For fusion products, subsequent digestion of the fusion protein with an appropriate proteolytic enzyme releases the RSP-1 or RSP-2 protein sequences.

The proteins of the invention can be used to raise monoclonal antibodies specific for RSP-1 or RSP-2. The antibodies can be used for diagnosis of malarial infection or as therapeutic agents. As therapeutic agents, monoclonal antibodies specific for RSP-1 and RSP-2 act on different points of Plasmodium infection: they can inhibit cytoadhesion of RSP-1 or RSP-2 positive erythrocytes, desequester RSP-1 or RSP-2 positive erythrocytes from endothelial cells, inhibit invasion of erythrocytes by merozoites and facilitate the removal of RSP-1 or RSP-2 positive erythrocytes from the blood by the spleen. The production of monoclonal antibodies against a desired antigen is well known to those of skill in the art. The multitude of techniques available to those skilled in the art for production and manipulation of various immunoglobulin molecules can thus be readily applied to inhibit binding. As used herein, the terms “immunoglobulin” and “antibody” refer to a protein consisting of one or more polypeptides substantially encoded by immunoglobulin genes. Immunoglobulins may exist in a variety of forms besides antibodies, including for example, Fv, Fab, and F(ab)₂, as well as in single chains.

Antibodies which bind the proteins of the invention may be produced by a variety of means. The production of non-human monoclonal antibodies, e.g., murine, lagomorpha, equine, etc., is well known and may be accomplished by, for example, immunizing the animal with a preparation containing the polypeptide. Antibody-producing cells obtained from the immunized animals are immortalized and screened. Methods of producing polyclonal and monoclonal antibodies are known to those of skill in the art. See, e.g., Coligan (1991) Current Protocols in Immunology Wiley/Greene, N.Y.; and Harlow and Lane (1989) Antibodies: A Laboratory Manual Cold Spring Harbor Press, N.Y. Specific monoclonal and polyclonal antibodies will usually bind with a Kd of at least about 0.1 mM, more usually at least about 1 μM, and most preferably at least about 0.1 μM or better.

In addition, the antibodies which bind to RSP-1 and RSP-2 can be “humanized” in accordance with known procedures, for example, as described in U.S. Pat. Nos. 5,530,101; 5,585,089; 5,693,761; and 5,693,762, the contents of which are incorporated herein by reference.

In another aspect of the present invention, additional antibodies that bind to the same epitopes as the antibodies specifically identified herein can be obtained using standard methods. For example, the ability of a candidate antibody to block the binding of one of the specifically identified antibodies described herein would be indicative that the candidate antibody binds to the same epitope on the antigen. Similarly, compounds, substances, or antibody fragments that have a similar ability to bind to the same epitopes as the antibodies specifically described herein can be identified, isolated, and used in the same way. In a preferred aspect of this method of identifying those antibodies, antibody fragments and/or compounds, those antibodies, antibody fragments and/or compounds should also have the ability to inhibit the cytoadhesion of RSP-1 or RSP-2 positive erythrocytes, to desequester RSP-1 or RSP-2 positive erthrocytes from endothelial cells during the appropriate stage of the parasite's life cycle and in another embodiment, facilitate the clearance of the RSP-1 or RSP-2 positive erythrocytes.

For example, the hybridoma Pf26G1/B4 deposited at the Collection Nationale de Cultures de Microorganismes (“C.N.C.M.”, INSTITUT PASTEUR, 25, Rue de Docteur Roux, F-75724, PARIS CEDEX 15) on Feb. 23, 2001 under the accession number I-2635 produces an antibody which is specific to chondroitin-sulphate A (CSA) cytoadhesion phenotype. The hybridoma secretes B4 monoclonal antibodies which react with the native P. falciparum proteins at the surface of ring-infected erythrocytes but not with the mature trophozoite and schizonte infected erythrocytes. B4 inhibits the cytoadhesion of ring-stage infected erythrocytes and also the re-invasion of the erythrocytes by the merozoites. Additional examples of antibodies include the C10 and D10 antibodies that bind to RSP-2. The hybridoma Pf26G1/C10 deposited at the Collection Nationale de Cultures de Microorganismes (“C.N.C.M.”, INSTITUT PASTEUR, 25, Rue de Docteur Roux, F-75724, PARIS CEDEX 15) on Feb. 23, 2001 under the accession number I-2636 produces the antibody C10.

The proteins and polynucleotides of the invention can be used in diagnostic applications for the detection of Plasmodium parasites or nucleic acids in a biological sample. The presence of parasites can be detected using several well recognized specific binding assays based on immunological results. For example, labeled antibodies to polypeptides of the invention can be used to detect Plasmodium in a biological sample. Alternatively, labelled polypeptides of the invention can be used to detect the presence of antibodies to RSP-1 or RSP-2 in a biological sample. For a review of the general procedures in diagnostic immunoassays, see Basic and Clinical Immunology 7th Edition (D. Stites and A. Terr ed.) 1991.

In addition, modified polypeptides, antibodies or other compounds capable of inhibiting the interaction between RSP-1 and RSP-2 and erythrocytes can be assayed for biological activity. For instance, polypeptides can be recombinantly expressed on the surface of cells and the ability of the cells to bind erythrocytes can be measured as described below. Alternatively, peptides or antibodies can tested for the ability to inhibit binding between erythrocytes and Plasmodium and/or RSP-1 and/or RSP-2.

Cell-free assays can also be used to measure binding of RSP-1 or RSP-2 polypeptides, for example, the sample can be immobilized on a solid surface and binding of labeled RSP-1 or RSP-2 can be determined. Many assay formats employ labeled assay components. The labeling systems can be in a variety of forms. The label (detectable moiety) may be coupled directly or indirectly to the desired component of the assay according to methods well known in the art. A wide variety of labels may be used. The component may be labeled by any one of several methods. The most common method of detection is the use of autoradiography with ³H, ¹²⁵I, ³⁵S, ¹⁴C, or ³²P labeled compounds or the like. Non-radioactive labels include ligands which bind to labeled antibodies, fluorophores, chemiluminescent agents, enzymes, and antibodies which can serve as specific binding pair members for a labeled ligand. The choice of label depends on sensitivity required, ease of conjugation with the compound, stability requirements, and available instrumentation.

In the case of the use nucleic acids for diagnostic purposes, standard nucleic hybridization techniques can be used to detect the presence of the genes identified here, RSP-1 and/or RSP-2. If desired, nucleic acids in the sample may first be amplified using standard procedures such as PCR. Diagnostic kits comprising the appropriate primers and probes can also be prepared.

RSP-1 and RSP-2 are useful in therapeutic and prophylactic applications for the treatment of malaria. In addition, the antibodies that bind to RSP-1 and/or RSP-2 or those compounds that have a similar ability to bind to the same epitopes as the antibodies specifically described herein can be formulated in a composition, e.g., pharmaceutical composition, suitable for use as a blocking agent of Plasmodium sp. during an infection. In such a method, the antibody or antibodies would be delivered to the infected patient during the appropriate stage of the parasitic life cycle in the blood. For example, with antibodies which bind RSP-2, the antibodies can be used to block passage through the infective life cycle of the parasite by inhibiting merozoite invasion. In this method, if the antibody, antibody fragment and/or substances can block at least a part of the RSP-2 positive merozoite invasion, then a reduction in the Plasmodium infection would be deemed to provide some level of treatment against the infection, particularly relative to the absence of the treatment regimen. Antibodies which bind RSP-2 and have the capacity to inhibit the cytoadhesion of RSP-2 positive erythrocytes and desequester the RSP-2 positive erythrocytes from endothelial cells, can be used to decrease the microvascular sequesteration of RSP-2 positive erythrocytes. The sequesteration of infected erythrocytes in the cerebral microvasculature is considered to be the essential underlying pathological process leading to coma and death. In this method, antibodies which bind to RSP-2 and have the capacity to inhibit the cytoadhesion of RSP-2 positive erythrocytes and/or desequester the RSP-2 positive erythrocytes from endothelial cells, can be used to reverse severe Plasmodium falciparum malaria as cerebral malaria. In another embodiment, the antibodies binding to RSP-2 erythrocytes are likely to result in the clearance of those erythrocytes from the circulation during passage through the spleen by interacting with Fc receptors.

Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249:1527-1533 (1990). The compositions are suitable for single administrations or a series of administrations. When given as a series, inoculations subsequent to the initial administration are given to boost the immune response and are typically referred to as booster inoculations.

The pharmaceutical compositions of the invention are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration that comprise a solution of the agents described above dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient and more preferably at a concentration of 25%-75%.

For aerosol administration, the polypeptides are preferably supplied in finely divided form along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery.

The amount administered to the patient will vary depending upon what is being administered, the state of the patient and the manner of administration. In therapeutic applications, compositions are administered to a patient already suffering from malaria in an amount sufficient to inhibit spread of the parasite through erythrocytes and thus cure or at least partially arrest the symptoms of the disease and its complications. An amount adequate to accomplish this is defined as “therapeutically effective dose.” Amounts effective for this use will depend on the severity of the disease, the particular composition, and the weight and general state of the patient.

Alternatively, the polypeptides of the invention can be used prophylactically as vaccines. The vaccines of the invention contain as an active ingredient an immunogenically effective amount of the binding domain polypeptide or of a recombinant virus as described herein. The immune response may include the generation of antibodies; activation of cytotoxic T lymphocytes (CTL) against cells presenting peptides derived from RSP-1 and/or RSP-2, or other mechanisms well known in the art. See e.g. Paul Fundamental Immunology Second Edition published by Raven press New York (incorporated herein by reference) for a description of immune response. Useful carriers are well known in the art, and include, for example, thyroglobulin, albumins such as human serum albumin, tetanus toxoid, polyamino acids such as poly(D-lysine:D-glutamic acid), influenza, hepatitis B virus core protein, hepatitis B virus recombinant vaccine. The vaccines can also contain a physiologically tolerable (acceptable) diluent such as water, phosphate buffered saline, or saline, and further typically include an adjuvant. Adjuvants such as incomplete Freund's adjuvant, aluminum phosphate, aluminum hydroxide, or alum are materials well known in the art. The DNA or RNA encoding RSP-1 or RSP-2 may be introduced into patients to obtain an immune response to the polypeptides which the polynucleotide encodes.

Vaccine compositions containing the proteins, nucleic acids or viruses of the invention are administered to a patient to elicit a protective immune response against the polypeptide. A “protective immune response” is one which prevents or inhibits the spread of the parasite through erythrocytes and thus at least partially prevent the symptoms of the disease and its complications. An amount sufficient to accomplish this is defined as an “immunogenically effective dose.” Amounts effective for this use will depend on the composition, the manner of administration, the weight and general state of health of the patient.

After immunization the efficacy of the vaccine can be assessed by production of antibodies or immune cells that recognize the antigen, as assessed by specific lytic activity or specific cytokine production. One skilled in the art would know the conventional methods to assess the aforementioned parameters.

Having generally described this invention, a further understanding can be obtained by reference to certain specific examples which are provided herein for purposes of illustration only, and are not intended to be limiting unless otherwise specified.

### EXAMPLES

### Example 1

Parasites

The following parasite isolates were used in this study: Palo-Alto (FUP)1 (Gysin, J. & Fandeur, T. Saimiri sciureus (Karyotype 14-7) an alternative experimental model of Plasmodium falciparum infection. Am. J. Trop Med. Hyg. 32,461-467 (1983)), IPL/BRE1 (Robert, C. et al Chondroitin-4-sulphate (proteoglycan), a receptor for Plasmodium falciparum-infected erythrocyte adherence on brain microvascular endothelial cells. Res. Immunol. 146, 383-393 (1995)), FCR3 subpopulations panned on CSA, CD36 and ICAM-1 (Pouvelle, B. Fusai, T. Lepolard, C. & Gysin, J. biological and biochemical characteristics of cytoadhesion of Plasmodium falciparum-infected erythrocytes to chondroitin-4-sulfate. Infect. Immun. 66, 4950-4956 (1998)), two isolates desequestered using soluble CSA from infected human placentas (42^{CSA} and 939^{CSA3}) and a CSA-selected population of a field isolate collected from the peripheral bloodstream (A53^{CSA}). The IE were cultured in RPMI 1640 containing bicarbonate, glutamine, 0.2% glucose, 50 μM hypoxanthine, 10 μg/ml gentamicin and 10% human AB⁺ serum, containing O⁺ eythrocytes, at 37° C. in a humidified atmosphere containing 5% O₂, 5% CO₂ and 90% N₂. IE cultures were synchronized by selecting ring-stage parasites using multiple 5% sorbitol treatments until the parasites reinvaded erythrocytes within 4 h.

Selection by Panning

Subpopulations of the Palo-Alto (FUP)1, (PA), IPL/BRE1 (BRE1), FCR3 strains and of the A53 isolate were selected by three successive pannings of mature stage IE on cellular CSA as previously described (Pouvelle, B. Fusai, T. Lepolard, C. & Gysin, J. biological and biochemical characteristics of cytoadhesion of Plasmodium falciparum-infected erythrocytes to chondroitin-4-sulfate. Infect. Immun. 66, 4950-4956 (1998)) using SBEC 17 (Gay, F. et al, Isolation and characterization of brain microvascular endothelial cells from Saimiri monkeys. An in vitro model for sequestration of Plasmodium falciparum-infected erythrocytes. J. Immunol. Meth. 184, 15-28 (1995)). In addition, subpopulations of PA and FCR3 were selected by three successive pannings of mature stage IE on cellular CD36 or ICAM-1, using chondroitinated SBEC C2 and 3A (Pouvelle, B. Fusai, T. Lepolard, C. & Gysin, J. biological and biochemical characteristics of cytoadhesion of Plasmodium falciparum-infected erythrocytes to chondroitin-4-sulfate. Infect. Immun. 66, 4950-4956 (1998)). Highly synchronsized ring-stage IE cultures were panned on SBEC 1D expressing CSA, CD36 and ICAM-1, as previously described. The cells were washed extensively to remove non-cytoadherent IE and were then incubated in culture medium for 24 hours to allow ring-stage IE to mature. RBC were added and the cultures grown as previously described.

Cytoadhesion and Cytoadhesion Inhibition Assays

Gelatin-enriched preparations of mature-stage IE were resuspended at a concentration of 5×10⁶ IE/ml in cytoadhesion medium at pH 6.8. Cytoadhesion microassays were then performed on 12-well IFA slides (Institut Pasteur, Paris) as previously described (Pouvelle, B. Fusai, T. Lepolard, C. & Gysin, J. biological and biochemical characteristics of cytoadhesion of Plasmodium falciparum-infected erythrocytes to chondroitin-4-sulfate. Infect. Immun. 66, 4950-4956 (1998)).

Ring-stage cytoadhesion assays were performed with endothelial cells as described above and with placental cryosections as previously described (Gysin, J., Pouvelle, B., Le Tonqueze, M., Edelman, L. & Boffa, M. C., Chondroitin sulfate of thrombomodulin is an adhesion receptor for Plasmodium falciparum-infected erythrocyics. Mol. Biochem. Parasitol. 88, 267-271 (1997)), with 1 to 10% parasitaemia (a 1×10⁷ IE/ml suspension).

For cytoadhesion inhibition assays, the IE were incubated with SBEC in the presence of 2.5 μg/ml thrombospondin, 0.1 mg/ml soluble CSA, dermatan sulphate (CSB), chondroitin-6-sulphate (CSC), keratin sulphate (Ker), hyaluronic acid (HA), heparin (Hep) (Fluka, France), or with SBEC previously incubated for 1 hour at 37° C. with 1 U/ml chondrotinase ABC (Fluka, France), 1 to 10 U/ml heparinase III (Hepase III) (Sigma, France) or 5 μg/ml anti-CD36 FA6-152 Mab (gift from Dr. Edelman). Inhibition assays were also carried out in the presence of sera obtained from Senegalese and Cameroonian patients living in areas of endemic malaria. The sera were obtained from primigravida and multigravida women, a male adult and a child, absorbed onto O⁺ human blood and SBEC ID, and tested at a dilution of 1/20. The results were compared to those for cytoadhesion in the presence of a 1/20 dilution of a pool of control sera from volunteers who had never contacted malaria.

The protease sensitivity of ring-stage IE cytoadhesion was analyzed using 5 μl of packed PA^{CSA} IE (8 hours post-invasion). IE were incubated with 10 or 100 μg/ml of trypsin TPCK (Signa) or 100 μg/ml of α-chymotrypsin TLCK (Sigma) for 30 minutes at 37° C. The digestion was stopped by adding culture medium containing 10% human plasma. The cells were then washed in cytoadhesion medium and allowed to cytoadhere to SBEC 1D, as previously described, using untreated PA^{CSA} as a control.

Surface Immunolabeling of IE

100 μl of a pool of 5 sera from Senegalese and Cameroonian patients living in an area of endemic malaria was adsorbed onto 30 l of O⁺ human blood, once at 37° C. and once at room temperature. 5 μl of highly synchronized ring- or trophozoite-stage PA^{CSA} IE were incubated on ice for 45 minutes with 100 l of the pool of sera diluted 1/10 in cytoadhesion medium. The IE were washed three times in cytoadhesion medium and incubated for 45 minutes on ice with FITC-conjugated anti-human IgG (Sigma, F-6380). After a final wash, the IE were observed by EPR microscopy (CELLscan, Scanalytics, Billerica, Mass.¹⁷).

Surface Iodination and Metabolic Labeling of IE

Synchronized mature-stage IE previously selected on CSA and CD36 by the receptor panning procedure (Scherf, A. et al. Antigenic variation in malaria: in situ switching, relaxed and mutually exclusive transcription of var genes during intra-erythrocytic development in Plasmodium falciparum. Embo J. 17, 5418-5426 (1998)) were enriched to >75% by the gelatin technique and then diluted with fresh erythrocytes to obtain approximately 20% ring-stage forms at the next cycle. Surface iodination was performed using the lactoperoxidase method (Buffet, P. A. et al. Plasmodium falciparum domain mediating adhesion to chondroitin sulfate A: A receptor for human placental infection. Proc. Natl. Acad. Sci. U.S.A. 96, 12743-12748 (1999)), 7, 14, 21 and 32 hours after re-invasion. Metabolic labeling was performed by adding 2 mCi ³⁵S-methionine to a 5 ml culture flask at the late schizont-stage. The culture was stopped 14 hours after re-invasion. Sequential extraction with 1% Triton X-100 then 2% SDS was carried out, followed by protease treatment (TPCK-treated trypsin and chymotrypsin TLCK (Sigma, Lt. Louis) as previously described). Samples iodinated or metabolically labeled were separated on a 5%-17.5% gradient acrylamide gel, which was then dried and placed against Kodak Bio Max MS1 film. Prestained protein markers were purchased from Life Technologies, Gaithersburg, Md. and New England BioLabs Inc., Beverly, Mass.

Statistical Analysis

The results of IE adhesion, cytoadhesion and cytoadhesion inhibition assays are expressed as means±SE. The Mann-Whitney test was used to evaluate the statistical significance of data from cytoadhesion inhibition assays and to compare cytoadhesion levels.

TABLE 1
Cytoadhesion of IE_{S}^{CSA} to Saimiri brain endothelial cells ID
IE^{CSA} cytoadhesion during the first 2 hours of the cycle

| 1E | 4 H | 8 H | 12 H | 16 | 20 H |
|---|---|---|---|---|---|
| Laboratory strains | | | | | |
| PA^{CSA} | 64 ± 42 | 73 ± 59 | 122 ± 177 | | |
| Bre^{CSA} | 35 ± 15 | 38 ± 19 | 128 ± 59 | | |
| FCR^{CSA} | 278 ± 210 | 237 ± 124 | 86 ± 21 | | |
| Peripheral Blood ioslate | | | | | |
| A53^{CSA} | 46 ± 29 | 32 ± 21 | 53 ± 33 | | |
| Placential Isolates | | | | | |
| 42^{CSA} | 29 ± 26 | 28 ± 12 | 51 ± 37 | | |
| 939^{CSA} | 47 ± 38 | 57 ± 50 | 71 ± 37 | | |

Data are the mean number (± SD) of cytoadherent IE/mm² of SBEC 1D monolayer (mean of quadruplicate spots).
Nd: not done.
The shadowed values correspond to PfEMP1-mediated cytoadhesion.

Results

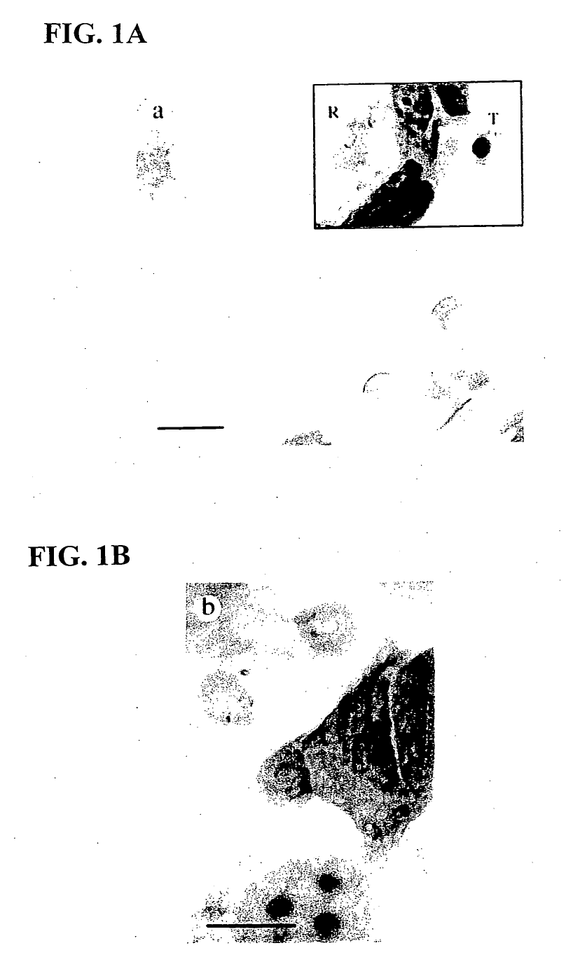

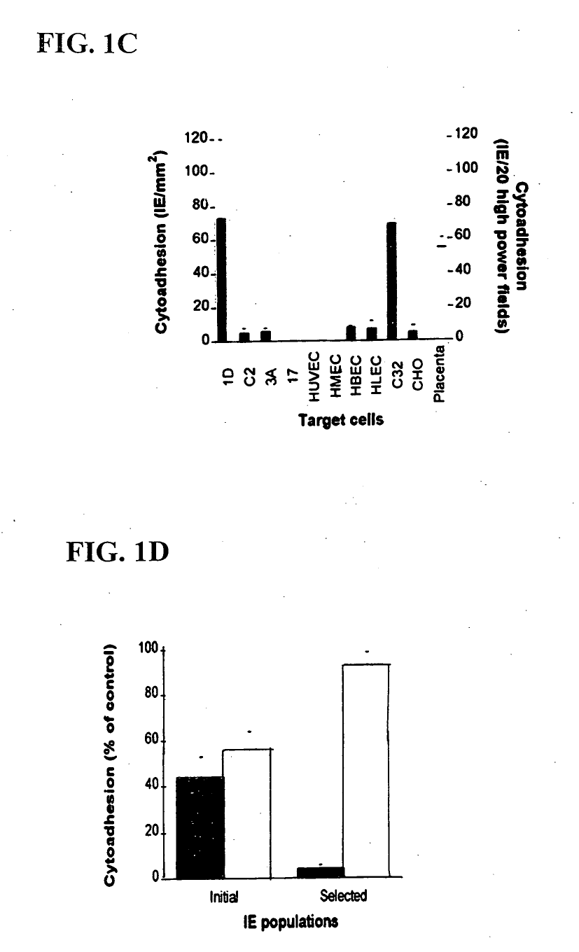

The peripheral blood of pregnant women infection with P. falciparum may be devoid of circulating ring-stage IE (Watkinson, M. & Rushton, D. J. Plasmodial pigmentation of placenta and outcome of pregnancy in West African moths. Brit. Med. J. 287, 251-254 (1983); Matteelli, A. et al, Malaria and anaemia in pregnant women in urban Zanzibar, Tanzania. Ann. Trop. Med. Parasitol. 88, 475-483 (1994)) or contain IE with little or no CSA binding phenotype (Fried, M. & Duffy, P. E. Adherence of Plasmodium falciparum to chondroitin sulfate A in the human placenta. Science 272, 1502-1504 (1996); Gysin, J., Pouvelle, B. Fievet, N., Schert, A. & Lepolard, C. Ex vivo desequestration of Plasmodium falciparum-infected erythrocytes from human placenta by chondroitin sulfate A. Infect. Immun. 67, 6596-6602 (1999)) despite the massive binding of IE to placenta CSA. This led us to investigate whether ring-stage IE from pregnant women are able to avoid circulating in the peripheral blood. We tested ring-stage adhesion to placenta and various cell types. Highly synchronized young ring-stage IE of P. falciparum isolate Palo-Alto (FUP) (Miller, L. H., Good, M. F. & Milton, G. Malaria pathogenesis. Science 264, 1878-1883 (1994)), prepared in vitro and selected for binding to CSA (PA^{CSA}), adhered to syncytiotrophoblasts in placenta cryosections and monolayers of cultured Saimiri brain endothelial cells (SBEC) (FIGS. 1A, B and C). Binding was observed immediately after merozoite reinvasion and continued throughout the ring-stage cycle (Table 1). There was extensive specific binding of ring-stage IE to SBEC ID and melanoma C32 cells (approx. 70 IE/mm²). The level of binding to human brain endothelial cells (HBEC), human lung endothelial cells (HLEC) and CHO cells was low but significant (≧5 IE/mm² of cell monolayer) (FIG. 1C). Finally, no ring-stage IE cytoadhesion to human umbilical vein endothelial cells (HUVEC, primo explants) and human dermal endothelial cells (HMEC-1) was detected. We investigated additional genetically different CSA-binding isolates: one from the peripheral blood of a child (A53^{CSA}), two placental isolates, 939^{CSA} and 42^{CSA}, and two isolates cultured in vitro (FCR3^{CSA} and Bre1^{CSA}). The results obtained confirmed that ring-stage IE bound to endothelial cells (Table 1).

Ring-stage adhesion was not detected in PA and FCR3 parasites selected for binding at the trophozoite stage to CD36 or ICAM-1 (<1 bound IE/mm² of SBEC 1D). These data suggest that the CSA-binding phenotype is connected to ring-stage adhesion by an unknown mechanism. We investigaged this by mixing equal numbers of highly synchronized ring-stage IE (8 hours after re-invasion) selected for bind to CSA and CD36 and allowing them to bind to a monolayer of SBEC 1D. Bound ring-stage IE were cultured and their phenotypes were assessed at the trophozoite stage by measuring the sensitivity to phenotype-specific inhibitors (soluble CSA mAb directed against CD 36) of adhesion. Almost all trophozoite binding was CSA-dependent whereas the non-selected IE combinations were inhibited similarly by both inhibitors (FIG. 1D). Similar selection was observed for the binding for mature IE to CSA if a mixture of IE subpopulations binding to CSA and ICAM-1 was tested (data not shown). Therefore, for the three phenotypes tested, the ability to cytoadhere before the trophozoite stage was strictly linked to the CSA-binding phenotype of mature IE.

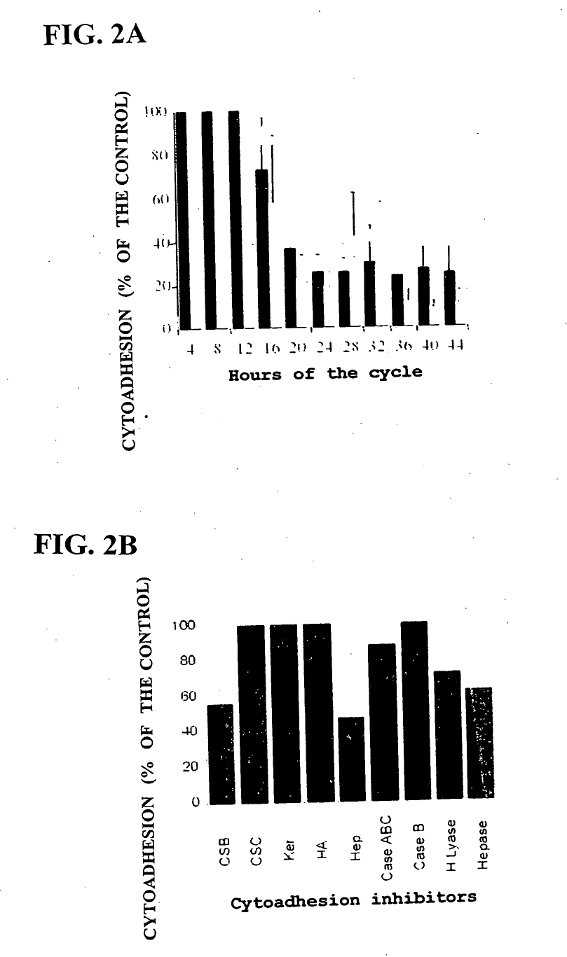

To identify the host receptor involved in ring-stage adhesion, we tested the inhibitory activity of CSA throughout the cycle. We observed that ring-stage IE^{CSA} cytoadhesion was insensitive to 0.1 mg/ml of CSA and to the prior treating of the target cells with 1 U/ml of chondroitinase ABC (Case ABC) until the 16th hour after invasion (FIG. 2A). At this time, with the beginning of knob formation and surface expression of the var^{CSA} gene, the inhibitory effects on binding of CSA and Case ABC were first seen. Inhibition was maximal at hour 24. Thus, all IE^{CSA} cytoadhere throughout the blood-stage cycle, switching from CSA-independent receptor interaction to a CSA-dependent phenotype 16 hours after invasion. We tested the possible involvement of various mature-stage IE adhesion receptors. Thrombospondin had no effect on IE binding to SBEC 1D (data not shown). Transfected CHO 745 (CSA⁻) expressing CD36, ICAM-1, VCAM or E-selectin at their surface showed non-specific cytoadhesion of ring-stage IE similar to that of the CHO-745 control cells (≦2 bound IEs/mm²). We also investigated the possible inhibitory effects of various glycosaminoglycans and their corresponding enzymes (FIG. 2B). The inhibition obtained with dermatan-sulphate (CSB) was not specific as Case ABC and B had no significant inhibitory activity. Hyaluronic acid had no activity whereas hyaluronate lyase had a low level of inhibitory activity, probably due to its secondary capacity to digest heparin and heparan sulphates. Heparin (100 μm/ml) and heparinase gave about 50% inhibition, SBEC ID do not express heparin at their surface. Instead, they express heparan sulphate proteoglycans (Fusai, T. et al, Characterisation of the chondroitin sulphate of Saimiri brain microvascular endothelial cells involved in P. falciparum cytoadhesion. Mol. Biochem. Parasitol., 108 (2000):25-37, and it is probably by completion with or digestion of SBEC 1D heparan sulphate that heparin and heparinase II partially inhibit ring-stage cytoadhesion. We are currently purifying SBEC 1D heparan sulphates to test their inhibition of ring-stage IE^{CSA} cytoadhesion.

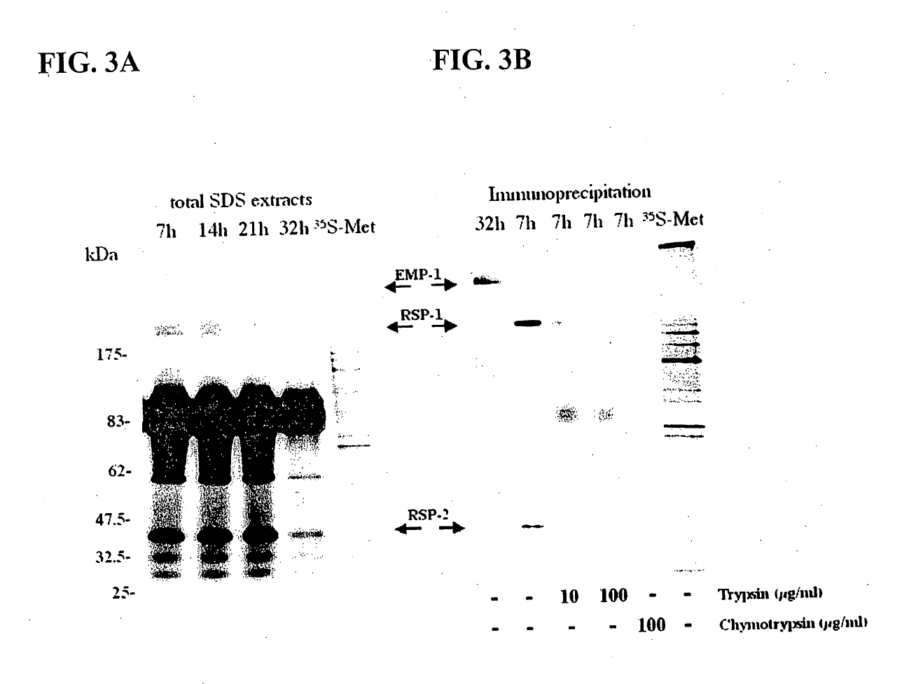

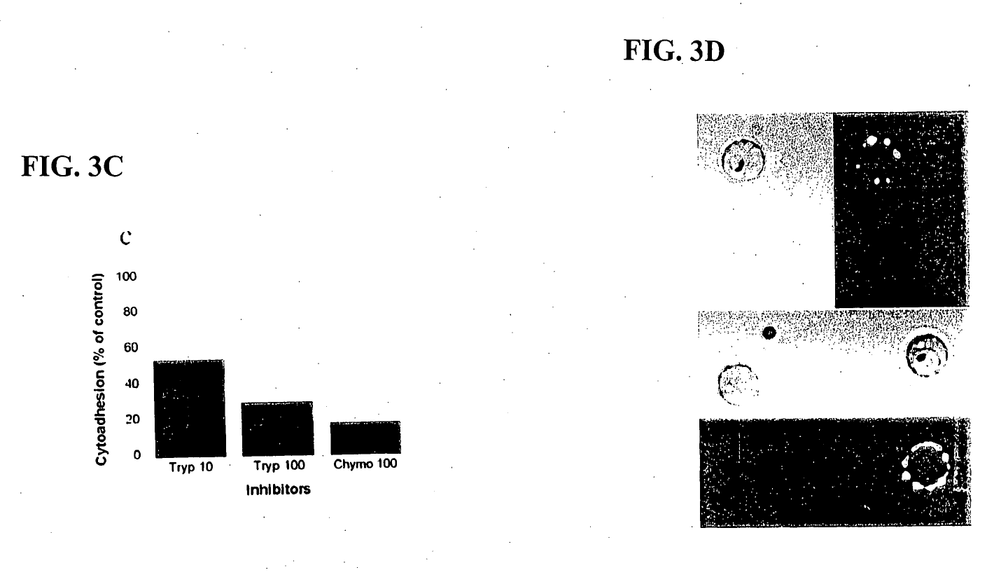

As ring-stage IE^{CSA} adhesion is not mediated by CSA, we thought it likely that a novel parasite surface molecule mediated binding to endothelial cells and syncytiotrophoblasts in early blood stage parasites. Surface iodination of FCR3^{CSA} ring-stage IE identified a molecule of approximately 200 kDa that was absent from control erythrocytes and is referred to here as “ring surface protein-1” (RSP-1) (FIG. 3). We immunoprecipitated surface-iodinated ring-stage IE extracts using a pool of serum from ultigravida women from Cameroon. The sera used recognized the 200 kDa molecule and a second molecule of approximately 40 kDa, termed RSP-2. RSP-2 was not detectable in total parasite extracts because it co-migrates with a band strongly labeled in uninfected erythrocytes. Parasite proteins identical in size to RSP-1 and RSP-2 were found in S-methionine labeled protein extracts from ring-stage IE. RSP-1 and RSP-2 were efficiently extracted in 2% SDS and were degraded by trypsin (100 μm/ml) or α-chymotrypsin (100 μg/ml) treatment (FIGS. 3A and B, and data not shown). Ring-stage IE adhesion was substantially inhibited at a protease concentration of 100 μg/ml (FIG. 3C), consistent with the involvement of RSP-1 and RSP-2 in the adhesion process. Both molecules were detected at the surface of young ring-stage IE but neither was present in mature trophozoites. In trophozoite IE, a large molecule, approximately 400 kDa in size, was detected at the IE surface (between 14 and 21 h post-invasion, FIGS. 3A and B) at a time coinciding with the switch in adhesive phenotype. The 400 kDa molecule of FCR3^{CSA} IE was identified in a previous study as the var gene product, which mediates the adhesion of mature forms to CSA. The RPS-1, RPS-2 and var^{CSA} molecule are naturally immunogenic and were efficiently immunoprecipitated by 8 sera from pregnant women (FIG. 3B and data not shown). These sera react with the surface of ring-stage and trophozoite-stage IE (FIG. 3E). Sera from malaria patients from Cameroon/Senegal (pregnant women, male adults and children) blocked the cytoadhesion of ring-stage IE to endothelial cells (FIG. 3F).

Our work challenges current views concerning the blood-stage biology of P. falciparum. It is generally accepted that ring-stage IE circulate in the blood and that adhesive properties become evident with the expression of the PfEMP1 molecule at the IE surface approximately 14 to 16 hours after invasion. Here we describe for the first time the specific adhesion of young ring IE to endothelial cells from critical target organs such as the brain and lung and to syncytiotrophoblasts. The differences between placental and peripheral blood parasitaemia and phenotype distribution observed in infected pregnant women can be accounted for by our findings. We suggest that the adhesion of ring-stage IE to placenta syncytiotrphoblasts precedes the CSA-binding of mature-stage IE, leading to a cryptic, or at least partially cryptic, life cycle of parasites with this adhesive phenotype. Evidence that ring-stage IE may cytoadhere in patients other than pregnant women comes from a recent study on sequestration of P. falciparum in the human brain (Silamut, K. et al. A quantitative analysis of the microvascular sequestration of malaria parasites in the human brain. Am. J. Pathol. 155, 395-410 (1999)). All developmental stages were observed in brain vessels of patients dying from cerebral malaria. Some vessels clearly contained large numbers of ring-stage IE but the nature of the interaction is unknown. As CSA is present in the brain microvasulature (Boffa, M. C., Jackman, R. W., Peyri, N. & George, B. Thrombomodulin in the central nervous system. Nouv. Rev. Fr. Hematol. 33, 423-429 (1991); Wong, V. L., Hofman, F. M. Ishii, H. & Fisher, M. Regional distribution of thrombomodulin in human brain. Brain Res. 556, 105 (1991); Gysin, J., Pouvelle, B., Le Tonqueze, M., Edelman, L. & Boffa, M. C., Chondroitin sulfate of thrombomodulin is an adhesion receptor for Plasmodium falciparum-infected erythrocyics. Mol. Biochem. Parasitol. 88, 267-271 (1997)), IE subpopulations may adhere to the same host cell throughout the blood stage cycle. Clearly, the absence or under-representation of specific virulent adhesive phenotypes in the bloodstream has a major impact on clinical studies based on peripheral blood-stage parasites. The role of ring-stage adhesion in tissue tropism should also be investigated. It is tempting to speculate that the massive accumulation of CSA-binding parasites observed in the placenta, for example, is due to the initial binding of rings.

The level of ring-stage adhesion to endothelial cells is markedly lower than that of trophozoite binding. This may be due to differences in the strength of the interaction between ligand and receptor pairs or to there being fewer ring-stage adhesion receptors than CSA molecules. Preliminary data obtained in flow-based assays, indicate an order to magnitude difference in strength of interaction between the ring and mature stages. Ring-stage adhesion is presumably maximal in the placenta, where blood flow is much lower than in other vascular beds.

The switch between two different adhesive phenotypes during the 48-hour blood-stage cycle is an entirely new phenomenon in the biology of P. falciparum. The expression pattern of IE surface molecules throughout the blood-stage cycle coincides with the observed change in adhesive phenotype, thus suggesting a role for RSP-1 and/or RSP-2 in ring-stage IE adhesion. In parasite that do not present ring-stage adhesion (CD36 phenotype), surface molecules with molecular masses similar to those of RSP-1 and RSP-2 were detected (data not shown). It is unclear whether RSP-1 and RSP-2 are members of a gene family or if phenotype-specific post-translational modifications of IE surface molecules (Fernandez, V. Hommel, M. chen, Q., Hagblom, P. & Wahlgren, M. Small, clonally variant antigens expressed on the surface of the Plasmodium falciparum-infected erythrocyte are encoded by the rif gene family and are the target of human immune responses. J. Exp. Med. 190, 1393-1404 (1999)) are responsible for the differences in adhesive features of rings.

Finally, the novel ring-stage IE surface molecules RSP-1 and RSP-2 are nature targets of the antibody-mediated immune response capable of blocking ring-stage adhesion. These antigens were therefore potential vaccine candidates that could reduce the severity of this major disease.

### EXAMPLE 2

The unique ability of P. falciparum infected erythrocytes (IE) to adhere to the endothelial cells of microvessels and placental syncytiotrophoblasts was previously attributed exclusively to mature stages (trophozoite and schizonts) of IE (Wahlgren, M., V. Fernandez, Q. Chen, S. Svard, and P. Hagblom. 1999. Waves of malarial variations Cell. 96:603-6). This dogma has been challenged by the recent discovery that ring-stage IE (rIE) can bind to endothelial cells and syncytiotrophoblasts, in a PfEMP1 independent manner, to an as yet unknown receptor (Pouvelle, B., P. A. Buffet, C. Lepolard, A. Scherf, and J. Gysin. 2000. Cytoadhesion of Plasmodium falciparum ring-stage-infected erythrocytess. Nat. Med. 6: 1264-1268). Adhesion of rIE is only seen in parasites that will acquire the CSA-binding phenotype at the trophozoite stage (a particular adhesion phenotype is seen in parasites that bind to placental syncytiotrophoblasts) (Scherf, A., B. Pouvelle, P. A. Buffet, and J. Gysin. 2001. Molecular mechanisms of Plasmodium falciparum placental adhesion. Cell Microbiol. 3: 125-31). This finding points to a specific subpopulation of IE that sequentially display two different adhesion phenotypes during the 48 hour blood stage cycle. This raises the possibility that non-circulating, and therefore cryptic parasite subpopulations, could be present in malaria patients. A previous study on sequestration of P. falciparum in the human brain supported the idea that ring-stage IE may cytoadhere in patients other than pregnant women and play a role in severe malaria (Silamut, K., N. H. Phu, C. Whitty, G. D. Turner, K. Louwrier, N. T. Mai, J. A. Simpson, T. T. Hien, and N. J. White. 1999. A quantitative analysis of the microvascular sequestration of malaria parasites in the human brain. Am J Pathol. 155:395-410). In this Example, all developmental stages were observed in brain vessels of patients dying from cerebral malaria and several vessels contained large numbers of rIE. Clearly, the under-representation of specific virulent adhesive phenotypes in the bloodstream may have a major impact on clinical studies based on peripheral blood-stage parasites.

Example 1 describes the presence of two novel ring-stage surface proteins named RSP-1 and RSP-2. Their expression during the blood-stage cycle coincides with the observed change in adhesive phenotype thus suggesting a role for RSP-1 and/or RSP-2 in rIE adhesion (see also; Pouvelle, B., P. A. Buffet, C. Lepolard, A. Scherf, and J. Gysin. 2000. Cytoadhesion of Plasmodium falciparum ring-stage-infected erythrocytes. Nat. Med. 6:1264-1268). In order to investigate this novel adhesive phenotype we developed mouse monoclonal antibodies (mAbs) that react specifically with intact rIE using a new immunization procedure (Lekana Douki, J. B., B. Traore, F. T. M. Costa, T. Fusaï, B. Pouvelle, Y. Sterkers, A. Scherf, and J. Gysin. 2002. Sequestration of Plasmodium falciparum-infected erythrocytes to chondroitin sulfate A, a receptor for maternal malaria: monoclonal antibodies against the native parasite ligand reveal pan-reactive epitopes in placental isolates. Blood. 100:1478-1483 and U.S. Ser. No. 09/867,536). These monoclonal antibodies allowed us to identify RSP-2 as an important molecule in the adhesion process and revealed the way by which RSP-2 gets to the surface of rIE. An unexpected finding is that RSP-2 is found on a large number of normal erythrocytes (nE). In placental isolates, non-infected erythrocytes that carry RSP-2 on their surface show a comparable adhesion phenotype to rIE under flow conditions using endothelial cells and are significantly more resistant to shear stress than PfEMP1 mediated IE binding.

Material and Methods.

Parasites

We used strains BXII, HB3, FCBR, Suk, H, IBR, FCR3^{CSA} and 6 cameroonian clinical isolates (number 24, 42, 42DJ, 193 and 939) eluted from human placentas with a soluble CSA (Gysin, J., B. Pouvelle, N. Fievet, A. Scherf, and C. Lepolard. 1999. Ex vivo desequestration of Plasmodium falciparum-infected erythrocytes from human placenta by chondroitin sulfate A. Infect Immun. 67:6596-602). CSA or CD36 and ICAM-1 adhesive phenotypes were selected from the FCR3 strain by panning on the Saimiri monkey brain microvascular endothelial cells Sc17, ScC2, Scs3A4 as described elsewhere (Gay, F., C. Robert, B. Pouvelle, S. Peyrol, A. Scherf, and J. Gysin. 1995. Isolation and characterization of brain microvascular endothelial cells from Saimiri monkeys. An in vitro model for sequestration of Plasmodium falciparum-infected erythrocytes. J Immunol Methods. 184: 15-28). Cytoadhesion assays using SBEC D cell lines were performed as described (Pouvelle, B., P. Meyer, C. Robert, L. Bardel and J. Gysin. 1997. Chondroitin-4-sulfate impairs in vitro and in vivo cytoadherence of Plasmodium falciparum infected erythrocytes. Mol Med. 3:508-1). Parasites were grown under standard culture conditions (Pouvelle, B., P. Meyer, C. Robert, L. Bardel and J. Gysin. 1997. Chondroitin-4-sulfate impairs in vitro and in vivo cytoadherence of Plasmodium falciparum infected erythrocytes. Mol Med. 3:508-1), replacing the 10% human serum with 0.25% Albumax. (Life Technology, France). For some experiments parasites were synchronized (±2 hours) (if not otherwise mentioned) by repeated 5% sorbitol treatments.

Development of Monoclonal Antibodies

To elicit antibodies against parasite-specific surface proteins of rIE, we immunized Balb/c mice, which had been rendered B-cell immuno-tolerant against normal human O⁻ red blood cells, with synchronized rIE of the CSA phenotype (rIE^{CSA}) as described earlier (Lekana Douki, J. B., B. Traore, F. T. M. Costa, T. Fusaï, B. Pouvelle, Y. Sterkers, A. Scherf, and J. Gysin. 2002. Sequestration of Plasmodium falciparum-infected erythrocytes to chondroitin sulfate A, a receptor for maternal malaria: monoclonal antibodies against the native parasite ligand reveal pan-reactive epitopes in placental isolates. Blood. 100:1478-1483). Two days after the third boost, mice were sacrificed and spleen cells fused with P3U1 cells and distributed in 96 well flat bottom plates as described (Galfre, G., and C. Milstein. 1981. In Langone J and Van Vunakis H (eds), Methods in Enzymology, vol. 73. Academic Press, New York.:3-46; Kohler, G., and C. Milstein. 1975. Continuous cultures of fused cells secreting antibody of predefined specificity. Nature. 256:495-497). Screening for antibodies directed against the surface rIE^{CSA} was done by liquid immunofluorescence assays (L-IFA). Cloning by limited dilution and expansion of positive clones was performed as described elsewhere (Galfre, G., and C. Milstein. 1981. In Langone J and Van Vunakis H (eds), Methods in Enzymology, vol. 73. Academic Press, New York.:3-46; Kohler, G., and C. Milstein. 1975. Continuous cultures of fused cells secreting antibody of predefined specificity. Nature. 256:495-497).

Purification of monoclonal antibodies (mAb) by modified protein A (MAPS, Bio-Rad, USA) and isotyping with the ImmunoPure monoclonal antibody isotyping Kit-I (Pierce, Rockford, Ill., USA) was done following the manufacture's recommendations. Three mabs B4 (IgG2a), C10 (IgG2a) and D10 (IgG3) were selected for their functional differences and further characterized.

Polyclonal Human Anti-P. falciparum Sera Pool

19 individual immune sera from multiparous Senegalese women were analysed individually by L-IFA. Sera positive in surface IFA with rIE were pooled at equivalent parts to constitute a sera pool that immunoprecipitated RSP-1 and RSP-2 I¹²⁵ labelled extracts of rIE. The human sera were obtained from Dr. O. Garraud from the Pasteur Institute of Dakar, Senegal, in conformity with the procedure recommended by the ethical committee of the Pasteur Institute.

Immunofluorescence Assays

MAbs were assessed by L-IFA at +4° C. and on air dried (AD-IFA) parasites selected for binding to CSA, CD36 and ICAM-1 as described (Lekana Douki, J. B., B. Traore, F. T. M. Costa, T. Fusaï, B. Pouvelle, Y. Sterkers, A. Scherf, and J. Gysin. 2002. Sequestration of Plasmodium falciparum-infected erythrocytes to chondroitin sulfate A, a receptor for maternal malaria: monoclonal antibodies against the native parasite ligand reveal pan-reactive epitopes in placental isolates. Blood. 100:1478-1483). To assess the surface exposure of the antigen, rIEs were preincubated with 100 μg/ml trypsin or chymotrypsin before adding the mAbs (Pouvelle, B., P. A. Buffet, C. Lepolard, A. Scherf, and J. Gysin. 2000. Cytoadhesion of Plasmodium falciparum ring-stage-infected erythrocytes. Nat. Med. 6:1264-126). The dynamics of RSP-2 and PfEMP1^{CSA} expression was assessed by L-IFA using synchronized IE^{CSA} (±2 hours) incubated with 40 μg/ml DAPI. After washing, the IE were incubated with 10 μg/ml purified mAbs (B4, C10, D10), washed, and bound antibodies detected with the goat (Fab)′2 Alexa Fluor 488 labelled anti-mouse IgG as mentioned above. IE^{CSA} were incubated a second time for 30 min at 4° C. with 10 μg/ml purified anti-PfEMP-1^{CSA} mAbs B4 (Lekana Douki, J. B., B. Traore, F. T. M. Costa, T. Fusaï, B. Pouvelle, Y. Sterkers, A. Scherf, and J. Gysin. 2002. Sequestration of Plasmodium falciparum-infected erythrocytes to chondroitin sulfate A, a receptor for maternal malaria: monoclonal antibodies against the native parasite ligand reveal pan-reactive epitopes in placental isolates. Blood. 100:1478-1483) which were covalently labelled to Alexa Fluor 594 (Molecular Probes, USA) following the manufacture's recommendations, washed and then analysed.

The percentage of RSP-2 surface stained rIE and nE by the mAbs was assessed by L-IFA using synchronized FCR3^{CSA} cultures, 4 hours after reinvasion, at a parasitaemia of 5, 10 and 20%. Results from four individual experiments were expressed as the arithmetic mean±2SD of labelled rIE and nE versus total number of erythrocytes and of labelled rIE versus all rIE. For the AD-IFA, a monolayer of IE was washed twice with PBS pH7.2 and co-incubated for 30 min at +37° C. with culture supernatant or 0.1-10 μg/ml purified mAbs in the presence of 1 μg/ml DAPI. After washing, bound antibodies were revealed with the Alexa Fluor 488 labelled anti-mouse IgG. In all experiments, the IF staining was analysed with a Nikon E800 microscope and acquired with a Ddx Nikon camera. As negative controls, either P3U1 culture supernatant or unrelated mouse IgG isotypes (Sigma, France) were used.

Immunoprecipitation of ³⁵S-Methionin and ¹²⁵I Surface Labelled IE-Extracts

Synchronized mIE, previously selected by panning on Sc17, C2 and 3A4 for the CSA, CD36 and ICAM-1 respectively (Gay, F., C. Robert, B. Pouvelle, S. Peyrol, A. Scherf, and J. Gysin. 1995. Isolation and characterization of brain microvascular endothelial cells from Saimiri monkeys. An in vitro model for sequestration of Plasmodium falciparum-infected erythrocytes. J Immunol Methods. 184:15-28), were cultivated in the presence of 2 mCi ³⁵S-methionin, (Amersham, France). The culture was stopped at 0 h or 8 hours and proteins extracted using Triton X100. In some cases TPCK-trypsin and TLCK-α-Chymotrypsin (Sigma, USA) treatments were performed before the protein extractions.

¹²⁵I surface labelling of rIE (>10% parasitemia) was done as previously described (Baruch, D. I., J. A. Gormely, C. Ma, R. J. Howard, and B. L. Pasloske. 1996. Plasmodium falciparum erythrocyte membrane protein 1 is a parasitized erythrocyte receptor for adherence to CD36, thrombospondin, and intercellular adhesion molecule 1. Proc Natl Acad Sci USA. 93:3497-502). Extraction was sequential and done in PBS with protease inhibitors containing 1% Triton X-100 and 2% SDS. Extracts were immunoprecipitated with mAbs. The sera pool from multiparous Cameroonian women (as described (Gysin, J., B. Pouvelle, N. Fievet, A. Scherf, and C. Lepolard. 1999. Ex vivo desequestration of Plasmodium falciparum-infected erythrocytes from human placenta by chondroitin sulfate A. Infect Immun. 67:6596-60)) was used as positive control and unrelated mouse IgG of the same isotypes (Sigma, France) as a negative controls for mAbs. IgG immune-complexes were recovered by incubation with protein G sepharose (Amersham-Pharmacia, France) and separated by 5-10% gradient SDS-PAGE, dried and exposed on a Kodak MR film.

Western Blot of rIE Triton X-100 Extracts

Proteins from Triton X-100 rIE-extracts, were separated by 12.5% SDS-PAGE and transferred to nitro-cellulose membranes, blocked in 5% skimmed milk in PBS pH7.2 for 2 hours at room temperature. Anti-RSP-2 mAbs at 20 μg/ml were incubated at room temperature for 1 hour. Membranes were washed twice with 5% milk in PBS, and incubated for 1 hour with goat anti-mouse IgG (H+L)-AP-Conjugate (Bio-Rad, France). After two additional washes, proteins were revealed with Alkaline Phosphatase Conjugate substrate Kit (Bio-Rad, France).

Static Cytoadhesion Inhibition and Disruption Assays

Cytoadhesion inhibition and disruption assays were performed on SBEC 1D cells grown to confluence on 12-dot IFA slides as described elsewhere (Pouvelle, B., P. Meyer, C. Robert, L. Bardel and J. Gysin. 1997. Chondroitin-4-sulfate impairs in vitro and in vivo cytoadherence of Plasmodium falciparum infected erythrocytes. Mol Med. 3:508-18) by assessing the laboratory CSA-binding strains FCR3, FCBR, BXII, SUK, HB, IBR and the placenta isolates no. 24, 42,42DJ, 193 and 939. In short: 20 μl of a mixture of synchronized rIE with nE or mIE from corresponding cultures at a parasitaemia of 4-15% for a total 10⁷ IE/ml RPMI 1640, pH6.8 were layered per dot of confluent SBEC 1D cells. For the inhibition assay, 20 μl of each mAb at a concentration of 0.2 to 200 μg/ml was deposited simultaneously with rIE or IE and corresponding nE/dot for 2 hours at +37° C. For the disruption assay the same mAbs were added after a one hour preincubation of IEs and nEs with endothelial cells for an additional 1 hour. Unbound erythrocytes were removed by vigorously washing the slides in RPMI 1640, pH6.8. Bound IE and nE were fixed with 0.25% glutaraldehyde in PBS pH7.2. The inhibition and disruption of cytoadhesion was expressed as bound IE and nE/mm² of confluent endothelial cells in the presence of mAbs, in comparison to unrelated mouse IgG of the same IgG isotype (Sigma, USA).

Inhibition and Shear Stress Resistance of Adherent rIE and nE Under Flow Conditions

Different concentrations (0.2-200 μg/ml) of anti-RSP-2 mAbs were mixed with 10⁷/ml 8±2 hour old rIE and flowed through a microslide (VD/3530-050, Camlab, Cambridge, UK) over a confluent, 3 day old monolayer of SEC ID cells at 0.05 Pascals, which is the shear stress that mimics conditions in a placenta. Results are expressed as the arithmetic mean±SD of the percentage of inhibition of cytoadhesion in three individual experiments. As a control we used unrelated homologous mouse IgG.

To evaluate the resistance of increasing shear stresses, synchronized 4±2 hour old rIE and nE from the same culture, were passed through a microslide at a final hematocrit of 25% in RPMI 1640, pH 7 for 10 min at 0.05 Pascals over a 3 day old confluent Sc1D cell layer (Pouvelle, B., B. Traore, P. A. Nogueira, B. Pradines, C. Lépolard, and J. Gysin. 2002. Modeling of Plasmodium falciparum-infected erythrocyte cytoadhesion in microvascular conditions: Chondroitin-4-sulfate binding, a competitive phenotype. J Dis. Infect. (2003) 187:302). After rinsing with the same medium for 10 min at 0.05 Pascals to remove residual unbound erythrocytes, the flow rate was increased for 10 min each time to gradually obtain wall shear stresses from 0.05 Pascals to 2.6 Pascals. Normal blood circulation through a normal placenta corresponds to 0.05 Pascals while 0.1 Pascals corresponds to a wall shear stress in post venule capillaries (Cooke, B. M. S. J. Rogerson., G. V. Brown., and R. L. Coppel. 1996. Adhesion of malaria-infected red blood cells to chondroitin sulfate A under flow conditions Blood. 88:4040-4). Experiments were repeated 3 times and expressed as the arithmetic mean percentage±SD of bound rIE and nE for each shear stress increment.

Functional and Chronological Differentiation Between RSP-2 and PfEMP1^{CSA} in a Parasite Cytoadhesion Inhibition Assay Under Flow Conditions

To compare the resistance of rIE and mature IE at various shear stresses we used a new cytoadhesion assay on SBEC 1D and placental tissues (B Traore et al. manuscript in preparation). In short: Four serial 7 μm normal human placenta cryosections (each of approximately 4 mm×4 mm) of the maternal compartment were mounted on a microscopic slide 75-25 mm and fixed with 0.5% para-formaldehyde in PBS at pH 7.2, for 2 hours at room temperature. After washing several times with PBS and then with RPMI 1640 pH 6.8, the slide is mounted on a perfusing chamber (Immunetics, France). An equipart mixture of 8±4 hours old synchronized rIE^{CSA} (including nE from the same culture) and 34±4 hours old IE^{CSA} at a final hematocrit of 25% in RPMI 1640, pH 7.2 were then flow through the chamber for 10 min at 0.05 Pascals. After rinsing for 10 min with the medium at the same shear stress, 100 ug/ml of a 50 kDa CSA (Fluka, France) dissolved in RPMI or 200 ug/ml of mAbs was flushed through the chamber for 10 min at the same shear stress. Slides from 3 experiments were recovered and stained by Giemsa. Mature IE, rIE and nE were counted and the result expressed as arithmetic mean percentage of bound erythrocytes±SD/20 fields of 100× Oil magnification as described elsewhere (Gysin, J., B. Pouvelle, N. Fievet, A. Scherf, and C. Lepolard. 1999. Ex vivo desequestration of Plasmodium falciparum-infected erythrocytes from human placenta by chondroitin sulfate A. Infect Immun. 67:6596-602).

Merozoite Invasion Inhibition Assay

Inhibition of merozoite invasion by mAbs was assessed by slightly modifying a technique described elsewhere (Pradines, B., A. Spiegel, C. Rogier, A. Tall, J. Mosnier, T. Fusai, J. F. Trape, and D. Parzy. (2000) Antibiotics for prophylaxis of Plasmodium falciparum infections: in vitro activity of doxycycline against Senegalese isolates. Am J Trop Med Hyg. 62:82-5). 200 μl/well of a mIE highly synchronised suspension (2.2% final hematocrit in RPMI+0.5% Albumax) were incubated in the above mentioned culture conditions in 96 flat bottom well-plates (NUNC, France), with 100 μl of 9.5-300 μg/ml mAbs. 1 μCi of ³H-hypoxanthine with a specific activity of 14.1 Ci/mmol (NEN Products, Dreiech, Germany) were added 4 h after invasion. 24 h later, cultures were freeze/thawed to lyse the erythrocytes and the contents of each well were collected on standard filter microplates (Unifilter® GF/B; Packard Instrument Company, Meriden, Conn.) and washed using a cell harvester (FilterMat® Cell harvester; Packard Instrument Company). Filters were dried and 25 μl of scintillation cocktail (Microscint®, Packard Instrument Company) was added to each well. Radioactivity of parasites was analysed with a scintillation counter (Top Count, Packard Instrument Company). As a positive control we used 1M Chloroquine and as a negative control unrelated mouse IgG of the same isotype. The percentage of inhibition was calculated using the t-test by comparing the cpm values of controls with those obtained in the presence of mAbs.

Results

Generation of Specific Monoclonal Antibodies Against the Surface of rIE

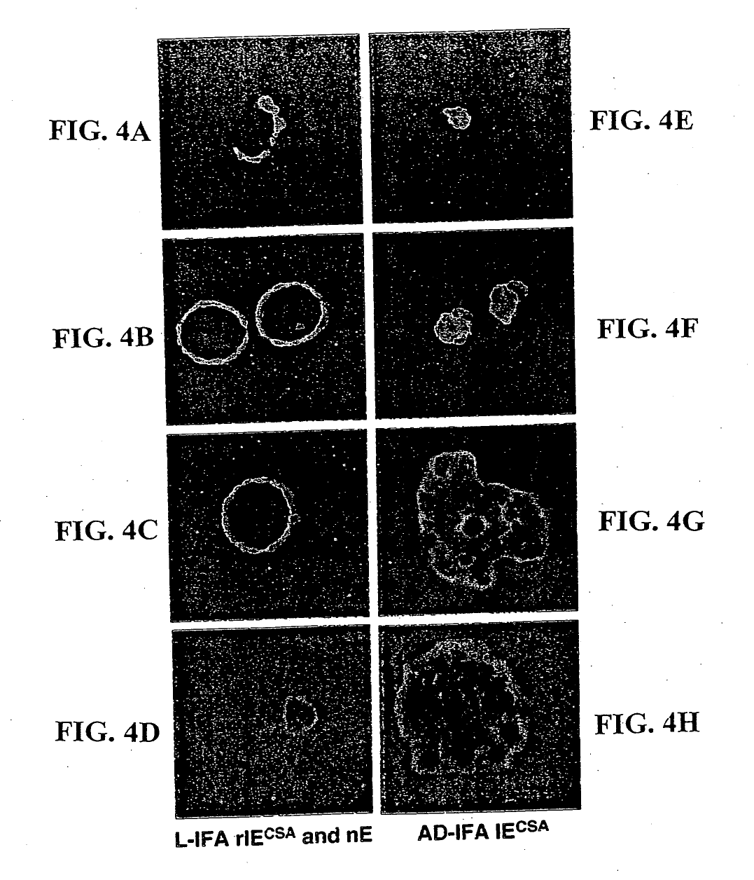

Mice were immunised with rIE from the FCR3 strain (selected for CSA-binding) using a novel immunisation procedure (see material and methods). Animals that responded to rIE surface antigens using L-IFA were chosen to produce mAbs. Three mAbs were obtained that recognize a parasite surface antigen present on rIE and inhibit adhesion of rIE to endothelial cells. MAbs B4 (IgG2a), C10 (IgG2a) and D10 (IgG3) were used to study the molecule(s) involved in rIE adhesion. The antigen was detectable at the surface of ring-stages immediately after merozoite release (FIG. 4A), whereas no labelling was seen in mature parasite stages (>20 hours) (FIG. 4D). However, mAbs B4, C10 and D10 reacted only with about 30% of rIE using L-IFA. AD-IFA results indicated that a fraction of the antigen is internalized during merozoite invasion (FIG. 4E) and de novo expression occurs during the early trophozoite-stage (FIG. 4F). Later, in the schizont stage, the antigen accumulates in rhoptries, where it can be identified by its typical double dot staining (FIG. 4G). This was confirmed using a rhoptry-specific marker (Rap1, data not shown). At the very end of the blood stage cycle before red blood cell disruption and merozoite release, the target of the mAbs seems to be translocated to the merozoite surface (FIG. 4H).

Surprisingly, normal erythrocytes (nE) were also stained by all three mAbs (FIG. 4B). At elevated parasitemia (5-10% of the whole erythrocytes population are infected) approximately 30% of all erythrocytes were positive using L-IFA. Surface labelling of rIE and nE was completely abolished by trypsin or chymotrypsin treatment (data not shown). Performing L-IFA with synchronized infected erythrocytes (E) at a 6 hours interval over the entire blood-stage cycle, revealed that the antigen was initially discharged on the erythrocyte membrane surface during contact with a merozoite (FIG. 4C). The antigen is then dispersed, probably by lateral movement, over the entire surface of rIE and nE. We concluded that the presence of the same antigen on nE is a direct consequence of aborted merozoite invasion. We addressed the question whether this rIE surface antigen could be released before the attachment of the merozoite to the erythrocyte membrane. To this end, we co-cultured synchronized schizont parasite stage (CD36, ICAM-1 and CSA adhesion phenotypes) for 18 hours in a two chamber culture system separating nE from synchronised late-stage infected erythrocytes by a membrane that allows diffusion of large proteins. Under such culture conditions we were not able to detect a signal on nE using mAbs B4, C10 and D10 (not shown). This demonstrates that contact is necessary for transfer of the antigen.

MAbs Detect a Protease-Sensitive 42 kDa Surface Protein that Forms a Complex with Two Other Proteins

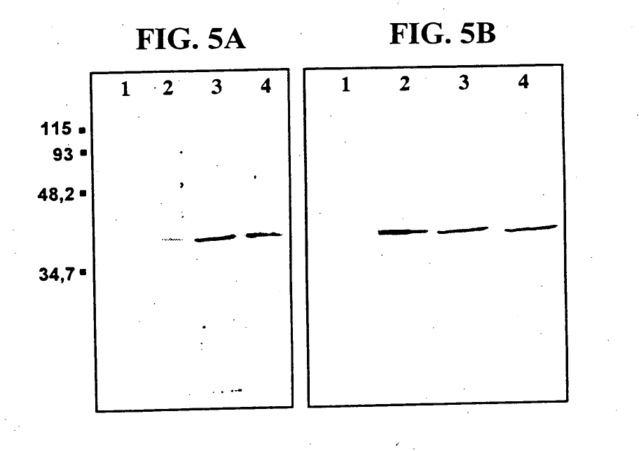

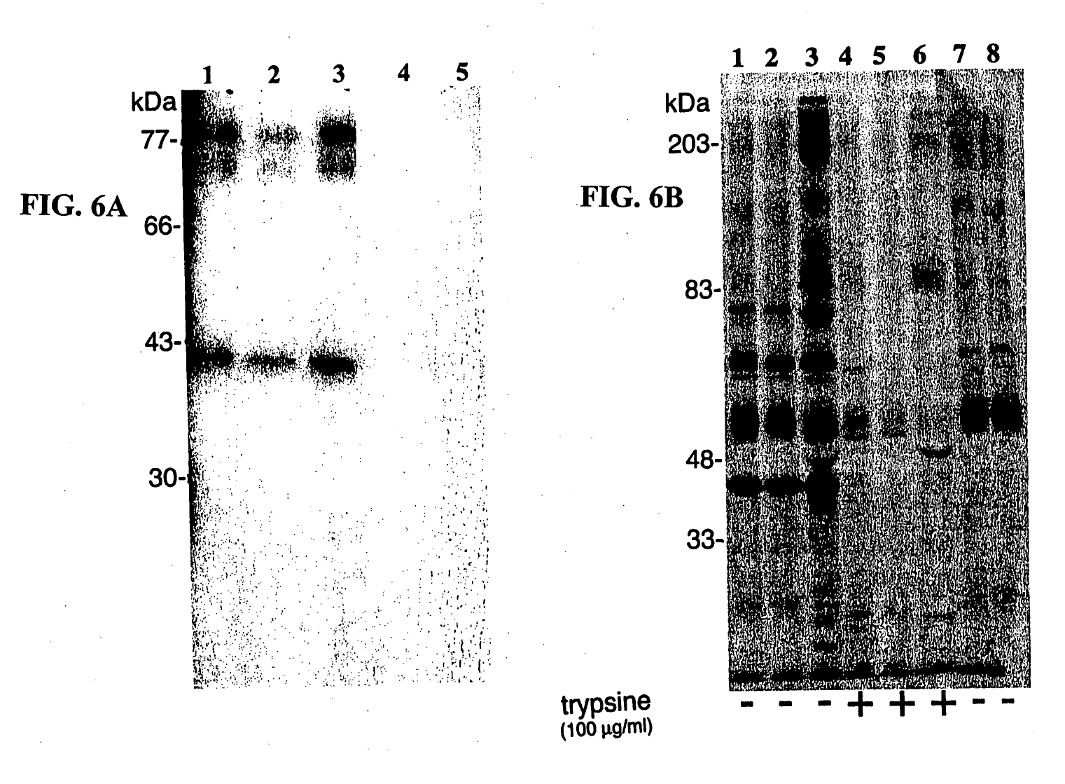

Western blot analysis using Triton-X100 parasite protein extracts (schizonts-stage IE and rIE) indicated that each mAb (B4, C10 and D10) recognised a 42 kDa protein (FIG. 5A). A molecule of identical size was detected by SDS-PAGE in FCR3 parasite extracts of the adhesion phenotypes CSA, CD36 and ICAM-1 (FIG. 5B). The mAbs immunoprecipitated proteins of molecular weights of approximately 79, 72 and 42 kDA (FIG. 6A) from S³⁵methionin labelled Triton X-100 rIE extracts. The same set of proteins was recognized by a sera pool from multiparous Senegalese women and was also immunoprecipitated from merozoite extracts of different adhesion phenotypes (CSA, CD36 and ICAM-1)(data not shown).

MAbs B4, C1 and D10 immunoprecipitated 3 proteins from ¹²⁵I-surface labelled rIE extracts. A major band was seen at approx. 42 kDa and two weaker bands of approx. 79 and 72 kDa. MAb B4 is shown as an example in FIG. 6B, (lanes 1 and 2). A pool of immune sera from multiparous Senegalese women immunprecipitated an additional band (doublet) of approx. 200 kDa (lane 3), which we had described earlier and corresponds to RSP-1 probably. These proteins are trypsin sensitive at similar concentrations described for RSP-1 and RSP-2 (Pouvelle, B., P. A. Buffet, C. Lepolard, A. Scherf, and J. Gysin. 2000. Cytoadhesion of Plasmodium falciparum ring-stage-infected erythrocytes. Nat. Med. 6: 1264-1268) (lanes 4, 5 and 6).

In conclusion, our results show that mAbs B4, C10 and D10 are specifically directed against a protein that has the characteristic features of RSP-2 (Pouvelle, B., P. A. Buffet, C. Lepolard, A. Scherf, and J. Gysin. 2000. Cytoadhesion of Plasmodium falciparum ring-stage-infected erythrocytes. Nat. Med. 6: 1264-1268). The absence of the 79 and 72 kDa protein in Western blots suggests that these proteins form a complex with the 42 kDa protein in the rhoptries, which is disrupted by SDS. A similar RSP-2 protein complex is observed at the surface of rIE. The identity of the 79 and 72 kDa proteins remains unkown.

Inhibition of Cytoadhesion in RSP-2 Positive rIE and nE

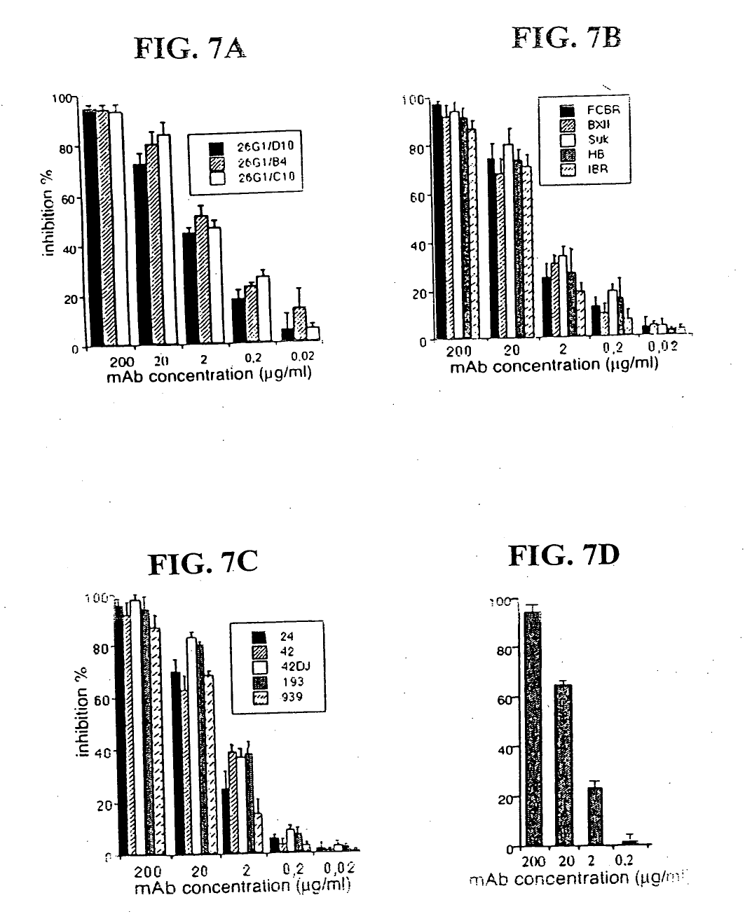

To confirm the implication of RSP-2 in cytoadhesion of rIE and nE, we used cytoadhesion inhibition and dissociation assays under static and flow conditions using the Saimiri brain endothelial cell line Sc1D and placenta cryosections (Gysin, J., B. Pouvelle, N. Fievet, A. Scherf, and C. Lepolard. 1999. Ex vivo desequestration of Plasmodium falciparum-infected erythrocytes from human placenta by chondroitin sulfate A. Infect Immun. 67:6596-602). Only RSP-2 positive rIE and nE of parasites selected for the CSA phenotype were able to cytoadhere to endothelial cells or syncytiotrophoblasts under static and flow conditions. The mAbs inhibited cytoadhesion of these synchronized rIE and nE by >94% at a concentration of 200 μg/ml and was still significant (approx. 50%) at a concentration of 2 μg/ml (FIG. 7A). The enzymatic digestion of RSP-2 with trypsin or chymotrypsin abolished this adhesion capacity completely (not shown). This result was in agreement with the L-IFA and immunoprecipitation results using ¹²⁵I labelled membrane extracts of rIE and nE. It clearly highlights the involvement of RSP-2 as the cytoadhesion ligand for the rIE CSA phenotype. In addition, the mAbs inhibited the adhesion of rIE CSA phenotypes equally well from five genetically distinct laboratory strains (FIG. 7B) and five distinct CSA binding parasite populations isolated from the placentas of five Cameroonian women (FIG. 7C). The inhibition of such rIE and nE cytoadhesion by mAbs was confirmed even when performing the inhibition assay under flow conditions at 0.05 Pa, which corresponds to shear stress observed in the placenta (FIG. 7D).

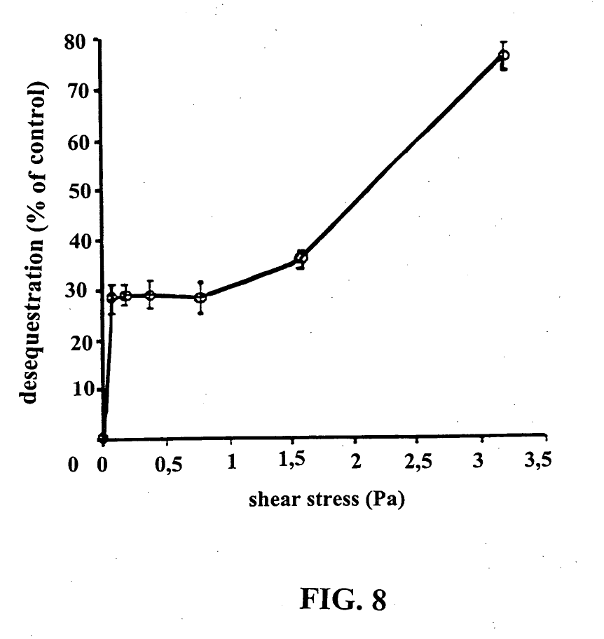

To establish that the interaction of RSP-2 with the host cell was not a low affinity or non specific binding event, we also performed a desequestration study by exposing adherent RSP-2 positive erythrocytes to increasing shear stresses. Synchronized RSP-2-tagged rIE and nE were passed over cryosections of normal human placentas at 0.05 Pa. The subsequent increase of the shear stress from 0.05 Pa to >3 Pa revealed an extremely strong interaction with syncytiotrophoblasts. Up to 50% of bound erythrocytes resisted a wall shear stress of around 2.1 Pa and about 25% of bound erythrocytes resisted to a wall shear stress exceeding 3 Pa at pH 6.8 (FIG. 8). This is roughly the same resistance to shear stress we found for mature IE of the CSA phenotype at pH 6.8, clearly indicating that RSP-2 adhesion resists much higher shear stress than the CD36 and ICAM-1 phenotypes (Pouvelle, B., B. Traore, P. A. Nogueira, B. Pradines, C. Lépolard, and J. Gysin. 2002. Modeling of Plasmodium falciparum-infected erythrocyte cytoadhesion in microvascular conditions: Chondroitin-4-sulfate binding, a competitive phenotype. J Dis. Infect. In press). Shear stress at such high levels are probably never encountered in malaria placentas or in the microvasculature. In conclusion, we show that RSP-2 is the molecule involved in ring stage cytoadhesion and our results also underline the possibility that this phenotype may sequester in target organs other than the placenta.

Chronology of RSP-2 and PfEMP1^{CSA} Surface Expression and Stage Specific Cytoadhesion Under Flow Conditions

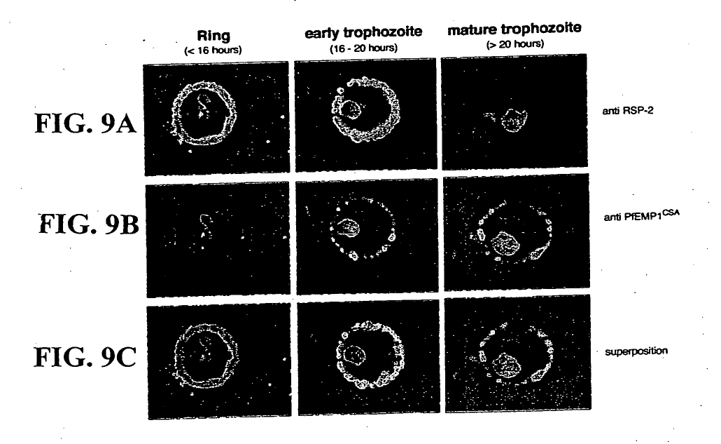

The dynamics of RSP-2 and PfEMP1^{CSA} expression was assessed by L-IFA using synchronized IE^{CSA} (±2 hours) incubated with 40 μg/ml of DAPI. After washing, the IE were incubated with 10 μg/ml purified mAbs B4, washed and bound antibodies detected with a goat (Fab)′2 Alexa Fluor 488 labeled anti-mouse IgG. IE^{CSA} were incubated a second time for 30 minutes at 4° C. with 10 μg/ml purified anti-PfEMP1 mAbs 1 B4/C4 (Lekana et al (2002):100(4):1478-83) which were covalently labeled to Alexa Fluor 594 (Molecular Probes, USA) following the manufacturer's recommendations, washed and then analyzed. FIG. 9A shows a RSP-2 positive staining (green) of the surface of rIE (<16 hours) and early trophozoites-stage IE (16-20 hours). No staining was detectable in mature trophozoites-stage IE (>20 hours). MAb directed against PfEMP1^{CSA} (panel B) shows specific surface staining (red) from early trophozoites on. Merging the panels A and B demonstrates a positive surface labelling of anti-RSP-2 and anti-PfEMP1^{CSA} on the same IE for a time period of approx. 4 hours (panel C). These results suggest that both adhesion ligands can be present on the same IE for at least a few hours during the switch from RSP-2 to PfEMP1^{CSA}. The sequential surface expression of RSP-2 then RSP-2 and PfEMP1^{CSA} followed by only PfEMP1^{CSA} during the parasite cycle provides the CSA-binding parasites with the capacity to remain sequestered during the whole blood-stage cycle.

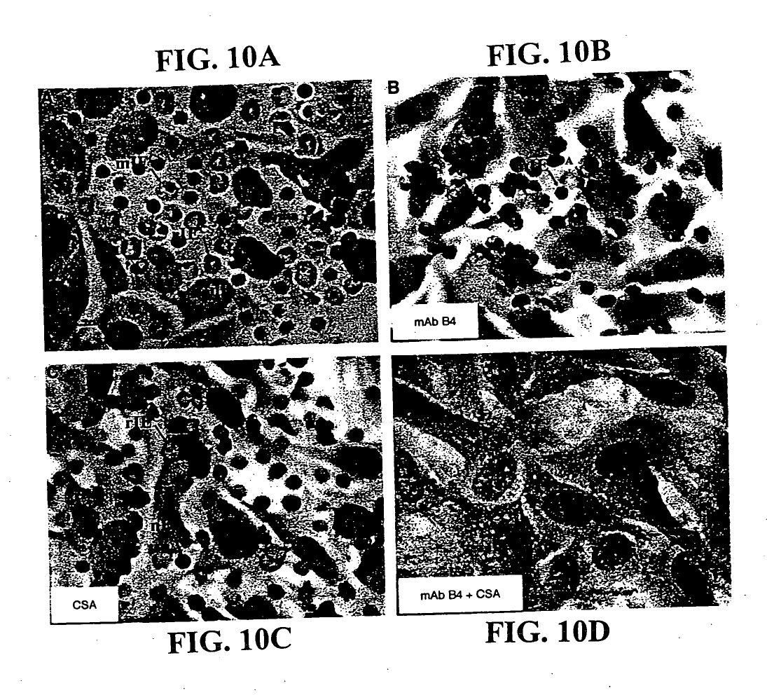

Adhesion to the same endothelial cells via two distinct parasite adhesion ligands was demonstrated using rIE^{CSA} and mature IE^{CSA} in adhesion assays using SBEC 1D under flow conditions at 0.05 Pa. Equal parts of rIE^{CSA}/nE^{CSA} and mature IE^{CSA} were passed over the endothelial cells. Both parasite populations bind to SBEC 1D at comparable numbers (FIG. 10A). Flushing with anti-RSP-2 B4 at 200 μg/ml resulted in the desequestration of rIE^{CSA}/nE but did not effect mature IE^{CSA} (FIG. 10B). Flushing with CSA (10 μg/ml final concentration) removed only the mature forms but not the rIE^{CSA}/nE (FIG. 10C). The combination of anti-RSP-2 and CSA eliminated all blood stage forms as shown in FIG. 10D.

Inhibition of Merozoite Invasion by mAbs

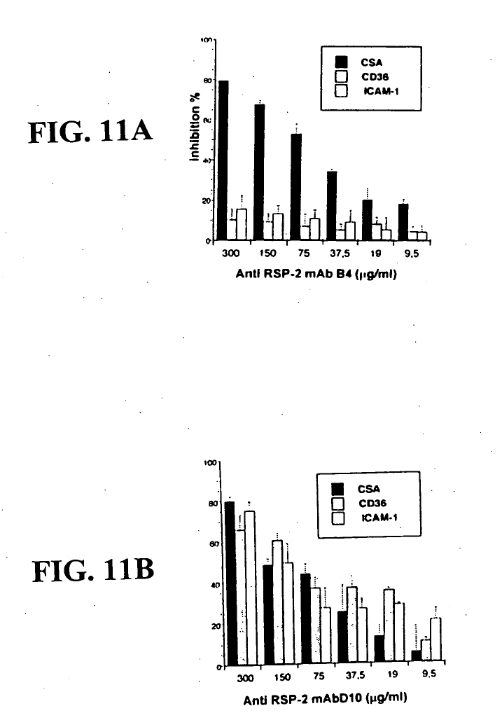

Since AD-IFA analysis indicates that RSP-2 seems to be on the merozoite surface at the end of schizogony, we suspected that anti-RSP-2 mAbs may also affect merozoite invasion. To address this question we incubated mAbs B4, C10 and D10 and unrelated IgG of the same isotype with highly synchronised IE (36 hours post invasion) and measured the incorporation of ³H hypoxanthine (for details see material and methods). The results indicated that mAb B4 efficiently inhibited merozoite invasion of the CSA phenotype but not of CD36 or ICAM-1 selected parasites (FIG. 11A), whereas mAb D10 inhibited the merozoite invasion of the phenotypes CSA, CD36 and ICAM-1 at similar levels (FIG. 11B). MAb C10 had no such inhibitory effect at high protein concentrations (not shown). The result indicated that the mAbs are directed against different epitopes on the RSP-2 molecule.

Discussion

We have developed mAbs that efficiently inhibit the adhesion of ring stage parasites. Two novel ring surface molecules (RSP-1 and RSP-2), which have been linked to rIE adhesion to endothelial cells and placenta syncytiotrophoblasts, have been identified. This challenges the prevailing dogma that only mIE are able to cytoadhere (Wahlgren, M., V. Fernandez, Q. Chen, S. Svard, and P. Hagblom. 1999. Waves of malarial variations Cell. 96:603-6). The mAbs recognize a trypsin sensitive and iodinatable parasite protein of approximately 42 kDa, which has similar features to the previously described RSP-2. None of the mAbs were directed against RSP-1, a 200 kDa molecule.

The synthesis of the mature IE surface adhesion molecule PfEMP1 begins very early during the ring stage and gets to the erythrocyte membrane in early trophozoites via a parasite specific intracellular trafficking pathway. One obvious question is, what mechanism does the RSP-2 use to be transported to the surface of rIE immediatly after the re-invasion process? We show that de novo synthesis of RSP-2 protein begins during the early trophozoite stage in the parasite endoplasmatic reticulum, from where the molecule is transported into the rhoptries. IFA analysis indicates that at least some protein material is translocated to the merozoite surface shortly before the rupture of the schizont. How it is transferred and dispersed over the entire erythrocyte surface is intriguing and needs further investigations. Furthermore, it is puzzling why RSP-2 progressively disappears at 16 to 20 hours in rIE and nE. This timing coincides with the appearance of PfEMP1 at the surface IE (Baruch, D. I., J. A. Gormely, C. Ma, R. J. Howard, and B. L. Pasloske. 1996. Plasmodium falciparum erythrocyte membrane protein 1 is a parasitized erythrocyte receptor for adherence to CD36, thrombospondin, and intercellular adhesion molecule 1. Proc Natl Acad Sci USA. 93:3497-502; Leech, J. H., J. W. Barnwell, L. H. Miller, and R. J. Howard. 1984. Identification of a strain-specific malarial antigen exposed on the surface of Plasmodium falciparum-infected erythrocytes. J Exp Med. 159:1567-75). Exposure of RSP-2 on the surface is confirmed by sensitivity to trypsin or chymotrypsin treated rIE and nE. Immunoprecipitation of extracts of surface iodinated nE and rIE using the anti-RSP-2 mAbs, revealed that RSP-2 is complexed with two other trypsin sensitive molecules. Although the molecular weights of the associated proteins on the surface changes slightly to those seen in the rhoptries, it is feasible that modifications occuring during the invasion process are involved.

RSP-2 is detectable only on the surface of a sub-population of rIE after the re-invasion process (about 30% of rIE). L-IFA with synchronised parasites showed that the transfer of RSP-2 from the merozoite membrane to the erythrocyte surface necessitates direct physical interaction. However, there appears to be no need to complete the invasion process. Experimental evidence shows that RSP-2 is not released into the medium before or during the merozoite invasion process. Normal red blood cells that were co-cultured with synchronized mIE in the same compartment separated by a permeable membrane did not acquire any detectable RSP-2 on the surface of nE (data not shown), confirming that contact is essential for the acquisition of the molecule. How the transfer of RSP-2 to the erythrocyte membrane occurs and why it is only associated with about 30% of rIE, whereas RSP-2 is carried into the erythrocyte in approximately 70% of rIE, remains puzzling and needs further investigation.

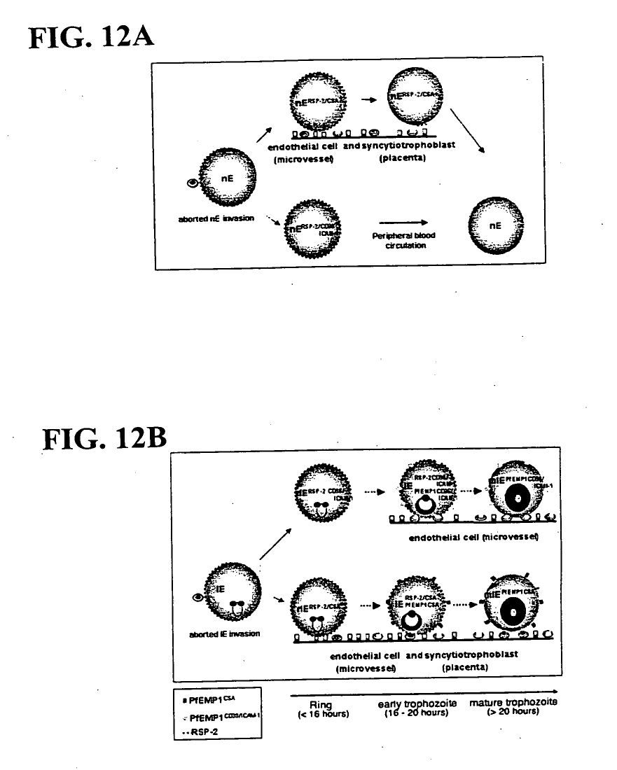

A novel finding is that the three mAbs B4, C10 and D10 also detect RSP-2 on the surface of nE. The percentage of RSP-2 tagged nE depends on the parasitemia. Up to 30% tagged nE are observed at elevated parasitemia (5 to 20%). From L-IFA studies with synchronized IE during the reinvasion process we assume that the presence of RSP-2 on the surface of nE and rIE is the consequence of aborted merozoite invasion. After an initial contact with the surface of erythrocytes, it appears that a large percentage of merozoites are not able to complete invasion process (a model is shown in FIG. 12). It remains unclear whether this is due to defective merozoites or phenotypic variation of merozoite surface molecules within a single schizont that are involved in the invasion of specific host cells, as has been illustrated for P. yoelii (Preiser, P. R., W. Jarra, T. Capiod, and G. Snounou. 1999. A rhoptry-protein-associated mechanism of clonal phenotypic variation in rodent malaria. Nature. 398:618-22).

We have found that the cytoadhesion of rIE is restricted to parasites that will express the CSA-binding phenotype later at the trophozoite stage (Pouvelle, B., P. A. Buffet, C. Lepolard, A. Scherf, and J. Gysin. 2000. Cytoadhesion of Plasmodium falciparum ring-stage-infected reythrocytes. Nat. Med. 6: 1264-1268). However, reactivity with mAbs strongly suggests the surface exposure of RSP-2 on rIE selected for CSA, CD36 and ICAM-1. Such a functional difference may reflect structural and/or conformational differences between RSP-2 molecules expressed in parasites with distinct PfEMP1 molecules. How this is linked to CSA-binding parasites is puzzling. One possible explanation could be that adhesive and non-adhesive forms of RSP-2 might be encoded by members of a gene family, that cross-react with mAbs B4, C10 and D10.

Anti-RSP-2 mAbs revealed that RSP-2 is present on the surface of rIE for approximately 16 to 20 hours after merozoite reinvasion, indicating a possible overlap of expression between RSP-2 and PfEMP1. This is supported by two independant experiments. First, anti RSP-2 and anti pfEMP1^{CSA} mAbs co-localise in young trophozoites for a short period (16-20 hours). Second, cytoadhesion of young trophozoites on endothelial cells or placenta cryosections cannot be inhibited by more than 25% with anti-RSP-2 or CSA, whereas the same reagents specifically inhibit cytoadhesion >90% in young rings (anti-RSP-2) or mature blood stages (CSA). We therefore concluded that both parasite-encoded surface ligands interact with different endothelial host receptors at the same time and that both ligands remained fully functional during this overlapping expression period (FIG. 12).

These data clearly imply that the sequential appearence of two distinct adhesion ligands can lead to parasite sequestration to the same host cell during the entire parasite life cycle. Our work demonstrates that placental syncitiotrophoblasts and some subpopulations of endothelial cells express the as yet unidentified RSP-2 receptor in addition to CSA. It can therefore be assumed that the CSA phenotype-IE have the capacity to propagate as a cryptic blood-stage in some tissues or organs. Ring stage mediated cytoadherence is likely to have important pathological consequences and may participate in the obstruction of blood vessels in the brain of fatal malaria patients (Silamut, K., N. H. Phu, C. Whitty, G. D. Turner, K. Louwrier, N. T. Mai, J. A. Simpson, T. T. Hien, and N. J. White. 1999. A quantitative analysis of the microvascular sequestration of malaria parasites in the human brain. Am J Pathol. 155:395-410).

Tagging RSP-2 positive red blood cells with antibodies is likely to result in their clearance from the circulation during passage through the spleen by interacting with Fc receptors. Another possible modification of antibody tagged erythrocytes is the increase in cell rigidity, as has been demonstrated recently. A mAb directed against glycophorin A rigidifies the erythrocyte membrane (Knowles, D. W., J. A. Chasis, E. A. Evans, and N. Mohandas. 1994. Cooperative action between band 3 and glycophorin A in human erythrocytes: immobilization of band 3 induced by antibodies to glycophorin A. Biophys J. 66:1726-32) and a similar modification might operate with RSP-2 antibodies. It is also possible that solely the presence of RSP-2 on the membrane surface renders erythrocytes less deformable which could be sufficient to result in their clearance by the spleen. Importantly, patient studies showed that nE become considerably more rigid in severe falciparum malaria (Dondorp, A. M., P. A. Kager, J. Vreeken, and N. J. White. 2000. Abnormal blood flow and red blood cell deformability in severe malaria. Parasitol Today. 16:228-32). It has also been suggested that parasites release substances during schizont rupture that could be involved in the rigidification of nE membranes in a reversible manner (Nauman, J., G. Adler, and M. Faryna. 1991. Eye-muscle membrane antibodies in autoimmune orbitopathy. Exp Clin Endocrinol.; 97:202-5). It is now possible to investigate whether RSP-2 plays a role in stiffening of nE in severe malaria patients using anti-RSP-2 mAbs as tools.

Massive removal of RSP-2 positive erythrocytes and particularly of nE that carry RSP-2 from the blood circulation is likely to cause severe anaemia in malaria patients. The destruction of antibody tagged nE is possible during the first 20 hours of each blood-stage cycle. RSP-2 positive nE can reach elevated levels of approximately 30% during in vitro culture at parasitemias of 5-10%. Interestingly, anaemia is a major factor of pathology observed in young children and primigravid women infected with P. falciparum in holoendemic areas (Shulman, C. E., W. J. Graham, H. Jilo, B. S. Lowe, L. New, J. Obiero, R. W. Snow, and K. Marsh. 1996, Malaria is an important cause of anaemia in primigravidae: evidence from a district hospital in coastal Kenya. Trans R Soc Trop Med Hyg 90:535⁻539). The implication of RSP-2 in anaemia is effectively supported by the presence of anti-RSP-2 antibodies in sera from pregnant women, adults and children (data not shown). We speculate that in the presence of anti-RSP-2 antibodies and high parasitemias, which are frequently observed in children and women during first pregnancy, anaemia might develop due to the elimination of RSP-2-tagged nE.

The fact that mAbs B4 and C10 inhibit merozoite invasion and recognize a 42 kDa molecule of a rhoptry complex, suggested that RSP-2 could correspond to a protein identified as RAP2 (Schofield, L., G. R. Bushell, J. A. Cooper, A. J. Saul, J. A. Upcroft, and C. Kidson.

1986. A rhoptry antigen of Plasmodium falciparum contains conserved and variable epitopes recognized by inhibitory monoclonal antibodies. Mol Biochem Parasitol. 18:183-95; Ridley, R. G., B. Takacs, H. Etlinger, and J. G. Scaife. 1990 A rhoptry antigen of Plasmodium falciparum is protective in Saimiri monkeys. Parasitology. 101:187-92) or RAP3 (Baldi, D. L., K. T. Andrews, R. F. Waller, D. S. Roos, R. F. Howard, B. S. Crabb, and A. F. Cowman. 2000. RAP 1 controls rhoptry targeting of RAP2 in the malaria parasite Plasmodium falciparum. EMBO J. 19:2435-43). It will be interesting to investigate how RAP2 and RAP3 relate to the multiple biological features, which are associated with the RSP-2 molecule during the blood stage cycle.

In conclusion, we have identified and characterized the molecule involved in rIE adhesion to different target organs involved in severe malaria and have detected a novel type of adhesion by non-infected erythrocytes. The newly acquired sticky phenotype of nE is mediated by a rhoptry-derived protein complex shed by merozoites to the erythrocyte surface during the invasion process. These findings are relevant for vaccine development, since antibodies against RSP-2 target important steps in parasite development such as merozoite invasion and the adhesion of rIE to host cells. Our data indicate that the same antibodies could also have a role in pathology by inducing anaemia. We now have, for the first time, the tools to investigate the role of a specific parasite molecule in the development of anaemia, which is a major factor of severe malaria.

Obviously, numerous modifications and variations on the present invention are possible in light of the above teachings. It is therefore to be understood that within the scope of the appended claims, the invention may be practiced otherwise than as specifically described herein.

1. An isolated antibody which binds to an protein, which mediates ring-stage Plasmodium falciparum infected erythrocytes cytoadhesion and is approximately 40 kiloDaltons in size as determined by SDS-polyacrylamide gel electrophoresis.

2. The isolated antibody of claim 1, which is a monoclonal antibody.

3. The isolated antibody of claim 1, which is a polyclonal antibody.

4. The isolated antibody of claim 1, which is a humanized antibody.

5. The isolated antibody of claim 1, which is the C10 antibody produced by the hybridoma Pf26G1/C10 deposited at the CNCM under the accession number I-2636.

6. (canceled)

7. Hybridoma Pf26G1/C10 deposited at the CNCM under the accession number I-2636.

8. A composition comprising the isolated antibody of claim 1 and a pharmaceutically acceptable carrier.

9. A method of treating a Plasmodium sp. infection in a patient, comprising administering the isolated antibody of claim 1 to the patient in an amount sufficient to desequester at least a part of erythrocytes, which express the protein on the cell surface and/or inhibit the invasion of erythrocytes by merozoites, which express the protein and/or to facilitate clearance of at least a part of erythrocytes, which express the protein by the spleen, in the patient.

10. The method of claim 9, wherein the antibody is the B4 antibody.

11. The method of claim 9, wherein the antibody is the C10 antibody.

12. (canceled)

13. A method of identifying a substance which binds to an epitope of RSP-2, which epitope is identified by the B4 antibody, the C10 antibody, or the D10 antibody, comprising contacting RSP-2 with the substance and determining whether the substance binds to the same epitope as the B4 antibody, the C10 antibody, or the D10 antibody, wherein the RSP-2 protein mediates ring-stage Plasmodium falciparum-infected cytoadhesion and is approximately 40 kildaltons in size as determined by SDS-polyacrylamide gel electrophoresis.

14. The method of claim 13, wherein the substance binds the same epitope as the B4 antibody.

15. The method of claim 13, wherein the substance binds the same epitope as the C10 antibody.

16. (canceled)

17. The method of claim 13, wherein the substance is an antibody.

18. A substance identified by the method of claim 13.

19. A composition comprising the substance of claim 18 and a pharmaceutically acceptable carrier.

20. A method of treating a Plasmodium sp. infection in a patient, comprising administering the substance of claim 18 to the patient in an amount sufficient to desequester at least a part of erythrocytes, which express the isolated protein on the cell surface and/or to inhibit the invasion of erythrocytes by merozoites, which express the protein and/or to facilitate clearance of at least a part of erythrocytes, which express the protein by the spleen, in the patient.

21. A method of diagnosing the Plasmodium falciparum blood-stage cycle in an individual suspected of being infected by Plasmodium falciparum comprising:
a. obtaining a biological sample from the individual;
b. contacting the biological sample with the isolated antibody of claim 1; and
c. identifying an interaction between the isolated antibody and an antigen in said biological sample, wherein the presence of an interaction indicates blood-stage infection.

22. A method of inhibiting cytoadhesion of erythrocytes and/or desequester erythrocytes from endothelial cells in a Plasmodium falciparum infected individual, comprising administering the isolated antibody of claim 1 in an amount to inhibit cytoadhesion of erythrocytes and/or desequester erythrocytes from endothelial cells, wherein the erythrocytes express the protein on the cell surface.

23. A method of tagging erythrocytes in a Plasmodium falciparum infected individual, comprising administering the isolated antibody of claim 1 in an amount to tag the erythrocytes thereby facilitating the clearance of the erythrocytes from the blood by the spleen, wherein the erythrocytes express the protein on the cell surface.
